(19) United States
(12) Patent Application Publication
(10) Pub. No.: US 2026/0137638 A1
(43) Pub. Date: May 21, 2026

(54) STAT3 INHIBITORS FOR USE IN THE TREATMENT OF NON-VIRAL LIVER CANCER

(71) Applicant: Tyardi Operating Company, Inc., Sugar Land, TX (US)

(72) Inventors: Imran Alibhai, Sugar Land, TX (US); Sofia de Achaval, Missouri City, TX (US)

(21) Appl. No.: 19/137,811

(22) PCT Filed: Dec. 11, 2023
(86) PCT No.: PCT/US2023/083440

§ 371 Date: Jun. 11, 2025

Related U.S. Application Data
(60) Provisional application No. 63/387,069, filed on Dec. 12, 2022.

(51) Int. Cl. A61K 31/18; A61K 31/44; A61K 31/47; A61K 39/00; A61P 35/00; C07K 16/22; C07K 16/28

(52) U.S. Cl. CPC A61K 31/18; A61K 31/44; A61K 31/47; A61P 35/00; C07K 16/22; C07K 16/2818; C07K 16/2827; A61K 2039/507; C07K 2317/24

(57) ABSTRACT

Provided herein, in some embodiments, are methods of treating a subject diagnosed with a non-viral liver cancer, the method comprising administering to the subject an effective amount of a STAT3 inhibitor.

### CROSS REFERENCE

This application claims the benefit of and priority to U.S. Provisional Patent Application No. 63/387,069, filed Dec. 12, 2022, which is incorporated by reference in its entirety herein.

### SUMMARY

Disclosed herein, in some embodiments, are methods of treating a subject diagnosed with a non-viral liver cancer, the method comprising administering to the subject an effective amount of a compound of Formula (I):

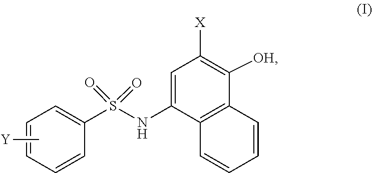

or a pharmaceutically acceptable salt thereof, wherein the variables are as defined herein.

In some embodiments, the compound of Formula (I) is N-(1′,2-dihydroxy-1,2′-binaphthalen-4′-yl)-4-methoxybenzenesulfonamide, N-(3,1′-Dihydroxy-[1,2′]binaphthalenyl-4′-yl)-4-methoxy-benzenesulfonamide, N-(4,1′-Dihydroxy-[1,2′]binaphthalenyl-4′-yl)-4-methoxy-benzenesulfonamide. N-(5,1′-Dihydroxy-[1,2′]binaphthalenyl-4′-yl)-4-methoxy-benzenesulfonamide, N-(6,1′-Dihydroxy-[1,2′]binaphthalenyl-4′-yl)-4-methoxy-benzenesulfonamide, N-(7,1′-Dihydroxy-[1,2′]binaphthalenyl-4′-yl)-4-methoxy-benzenesulfonamide, N-(8,1′-Dihydroxy-[1,2′]binaphthalenyl-4′-yl)-4-methoxy-benzenesulfonamide, 4-Bromo-N-(1,6′-dihydroxy-[2,2′]binaphthalenyl-4-yl)-benzenesulfonamide, or 4-Bromo-N-[4-hydroxy-3-(1H-[1,2,4]triazol-3-ylsulfanyl)-naphthalen-1-yl]-benzenesulfonamide, or a pharmaceutically acceptable salt thereof.

In some embodiments, the compound of Formula (I) is N-(1′,2-dihydroxy-1,2′-binaphthalen-4′-yl)-4-methoxybenzenesulfonamide, having the following structure:

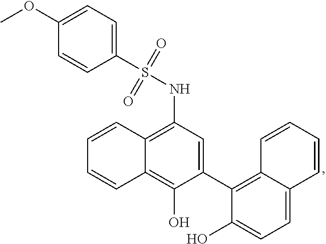 [SMILES: COc1ccc(S(=O)(=O)Nc2cc(-c3c(O)ccc4ccccc34)c(O)c3ccccc23)cc1]

or a pharmaceutically acceptable salt thereof.

Further disclosed herein, in some embodiments, are methods of treating a subject diagnosed with a non-viral liver cancer, the method comprising administering to the subject an effective amount of a compound of Formula (II):

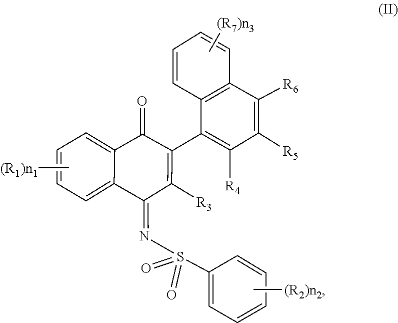

or a pharmaceutically acceptable salt thereof, wherein the variables are as defined herein.

Other objects and advantages will become apparent to those skilled in the art from a consideration of the ensuing Detailed Description, Examples, and Claims.

### BRIEF DESCRIPTION OF THE DRAWINGS

FIG. 1 shows exemplary RECIST scans in two non-viral HCC patients demonstrating durable partial responses.

FIG. 2 illustrates target tumor trajectory for Participant B on TTI-101 monotherapy.

FIG. 3 illustrates an excerpt of target and select non-target tumor trajectories for Participant B on TTI-101 treatment, and trends demonstrating potential resensitization to immune checkpoint inhibitor therapy.

FIG. 4 illustrates a partial response in a non-viral HCC patient.

### DETAILED DESCRIPTION

In some embodiments, the present disclosure provides methods of treating a subject diagnosed with a non-viral liver cancer, the method comprising administering to the subject an effective amount of a compound of Formula (I) or a compound of Formula (II), or pharmaceutically acceptable salts thereof.

### Definitions

As used herein the specification, “a” or “an” means one or more. As used herein, when used in conjunction with the word “comprising”, the words “a” or “an” means one or more than one. As used herein “another” means at least a second or more. Still further, the terms “having”, “including”, “containing” and “comprising” are interchangeable and one of skill in the art is cognizant that these terms are open ended terms. Some embodiments of the disclosure consist of or consist essentially of one or more elements, method steps, and/or methods of the disclosure. It is contemplated that any method, compound, or composition described herein can be implemented with respect to any other method, compound, or composition described herein.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide. 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like.

As used herein, “pharmaceutically acceptable excipient” refers to any substance in a pharmaceutical formulation other than the active pharmaceutical ingredient(s). Exemplary pharmaceutical excipients include those that aid the manufacturing process: protect, support or enhance stability; increase bioavailability; or increase patient acceptability. They may also assist in product identification or enhance the overall safety or function of the product during storage or use.

As used herein, a “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In some embodiments, the subject is a human. In some embodiments, the subject is a non-human animal. The terms “human,” “patient,” “subject,” and “individual” are used interchangeably herein. None of these terms require the active supervision of medical personnel.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or reverses or slows the progression of the disease, disorder or condition (also “therapeutic treatment”).

In general, the “effective amount” of a compound refers to an amount sufficient to elicit the desired biological response. As will be appreciated by those of ordinary skill in this art, the effective amount of a compound of the disclosure may vary depending on such factors as the desired biological endpoint, the pharmacokinetics of the compound, the disease being treated, the mode of administration, and the age, weight, health, and condition of the subject. A “therapeutically effective amount” of a compound is an amount sufficient to provide a therapeutic benefit (e.g., treating, preventing, and/or ameliorating cancer in a subject, or inhibiting protein-protein interactions mediated by an SH2 domain in a subject, at a reasonable benefit/risk ratio applicable to any medical treatment) in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent. A “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent. A “prophylactic treatment” contemplates an action that occurs before a subject begins to suffer from the specified disease, disorder or condition.

The term “inhibitor” as used herein, for example with respect to STAT3 inhibitors, refers to one or more molecules that interfere at least in part with the activity of STAT3 to perform one or more activities, including the ability of STAT3 to bind to a molecule and/or the ability to be phosphorylated.

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

The term “alkyl” refers to a straight or branched chain alkane (hydrocarbon) radical containing from 1 to 12 carbon atoms, preferably 1 to 6 carbon atoms. Exemplary “alkyl” groups include methyl, ethyl, propyl, isopropyl, n-butyl, t-butyl, isobutyl pentyl, hexyl, isohexyl, heptyl, 4,4-dimethylpentyl, octyl, 2,2,4-trimethylpentyl, nonyl, decyl, undecyl, dodecyl, and the like. The term “(C₁-C₄) alkyl” refers to a straight or branched chain alkane (hydrocarbon) radical containing from 1 to 4 carbon atoms, such as methyl, ethyl, propyl, isopropyl, n-butyl, t-butyl, and isobutyl. “Substituted alkyl” refers to an alkyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃. OCF₃, cycloalkyl, alkenyl, cycloalkenyl, alkynyl, heterocycle, aryl, ORa, SRa, S(═O)Re, S(═O)₂Re, P(═O)₂Re, S(═O)₂ORe, P(═O)₂ORe, NRbRc, NRbS(═O)₂Re, NRbP(═O)₂Re, S(═O)₂NRbRc, P(═O)₂NRbRc. C(═O)ORd, C(═O)Ra, C(═O)NRbRc, OC(═O)Ra, OC(═O)NRbRc, NRbC(═O)ORe, NRdC(═O)NRbRc, NRdS(═O)₂NRbRc, NRdP(═O)₂NRbRc, NRbC(═O)Ra, or NRbP(═O)₂Re, wherein each occurrence of Ra is independently hydrogen, alkyl, cycloalkyl, alkenyl, cycloalkenyl, alkynyl, heterocycle, or aryl; each occurrence of Rb, Rc, and Rd is independently hydrogen, alkyl, cycloalkyl, heterocycle, aryl, or said Rb and Rc together with the N to which they are bonded optionally form a heterocycle; and each occurrence of Re is independently alkyl, cycloalkyl, alkenyl, cycloalkenyl, alkynyl, heterocycle, or aryl. In the aforementioned exemplary substituents, groups such as alkyl, cycloalkyl, alkenyl, alkynyl, cycloalkenyl, heterocycle, and aryl can themselves be optionally substituted.

The term “alkenyl” refers to a straight or branched chain hydrocarbon radical containing from 2 to 12 carbon atoms and at least one carbon-carbon double bond. Exemplary such groups include ethenyl or allyl. The term “C₂-C₆ alkenyl” refers to a straight or branched chain hydrocarbon radical containing from 2 to 6 carbon atoms and at least one carbon-carbon double bond, such as ethylenyl, propenyl, 2-propenyl, (E)-but-2-enyl, (Z)-but-2-enyl. 2-methy(E)-but-2-enyl, 2-methy(Z)-but-2-enyl, 2,3-dimethy-but-2-enyl, (Z)-pent-2-enyl, (E)-pent-1-enyl, (Z)-hex-1-enyl, (E)-pent-2-enyl, (Z)-hex-2-enyl, (E)-hex-2-enyl, (Z)-hex-1-enyl, (E)-hex-1-enyl, (Z)-hex-3-enyl, (E)-hex-3-enyl, and (E)-hex-1,3-dienyl. “Substituted alkenvl” refers to an alkenyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORa, SRa, S(═O)Re, S(═O)₂Re, P(═O)₂Re, S(═O)₂ORe. P(═O)₂ORe, NRbRc, NRbS(═O)₂Re, NRbP(═O)₂Re, S(═O)₂NRbRc, P(═O)₂NRbRc, C(═O)ORd, C(═O)Ra, C(═O)NRbRc, OC(═O)Ra, OC(═O)NRbRc, NRbC(═O)ORe, NRdC(═O)NRbRc, NRdS(═O)₂NRbRc, NRdP(═O)₂NRbRc, NRbC(═O)Ra, or NRbP(═O)₂Re, wherein each occurrence of Ra is independently hydrogen, alkyl, cycloalkyl, alkenyl, cycloalkenyl, alkynyl, heterocycle, or aryl: each occurrence of Rb. Rc. and Rd is independently hydrogen, alkyl, cycloalkyl, heterocycle, aryl, or said Rb and Rc together with the N to which they are bonded optionally form a heterocycle; and each occurrence of Re is independently alkyl, cycloalkyl, alkenyl, cycloalkenyl, alkynyl, heterocycle, or aryl. The exemplary substituents can themselves be optionally substituted.

The term “alkynyl” refers to a straight or branched chain hydrocarbon radical containing from 2 to 12 carbon atoms and at least one carbon to carbon triple bond. Exemplary such groups include ethynyl. The term “C₂-C₆ alkynyl” refers to a straight or branched chain hydrocarbon radical containing from 2 to 6 carbon atoms and at least one carbon-carbon triple bond, such as ethynyl, prop-1-ynyl, prop-2-ynyl, but-1-ynyl, but-2-ynyl, pent-1-ynyl, pent-2-ynyl, hex-1-ynyl, hex-2-ynyl, or hex-3-ynyl. “Substituted alkynyl” refers to an alkynyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O). CF₃, OCF₃, cycloalkyl, alkenyl, cycloalkenyl, alkynyl, heterocycle, aryl, ORa, SRa, S(═O)Re, S(═O)₂Re, P(═O)₂Re, S(═O)₂ORe, P(═O)₂ORe, NRbRc, NRbS(═O)₂Re, NRbP(═O)₂Re, S(═O)₂NRbRc, P(═O)₂NRbRc, C(═O)ORd, C(═O)Ra, C(═O)NRbRc, OC(═O)Ra, OC(═O)NRbRc, NRbC(═O)ORe, NRdC(═O)NRbRc, NRdS(═O)₂NRbRc, NRdP(═O)₂NRbRc, NRbC(═O)Ra, or NRbP(═O)₂Re, wherein each occurrence of Ra is independently hydrogen, alkyl, cycloalkyl, alkenyl, cycloalkenyl, alkynyl, heterocycle, or aryl; each occurrence of Rb, Rc and Rd is independently hydrogen, alkyl, cycloalkyl, heterocycle, aryl, or said Rb and Rc, together with the N to which they are bonded optionally form a heterocycle; and each occurrence of Re is independently alkyl, cycloalkyl, alkenyl, cycloalkenyl, alkynyl, heterocycle, or aryl. The exemplary substituents can themselves be optionally substituted.

The term “cycloalkyl” refers to a fully-saturated cyclic hydrocarbon group containing from 1 to 4 rings and 3 to 8 carbons per ring. “C₃-C₇ cycloalkyl” refers to cyclopropyl, cyclobutyl, cyclopentyl, cyclohexyl, or cycloheptyl. “Substituted cycloalkyl” refers to a cycloalkyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃. OCF₃, cycloalkyl, alkenyl, cycloalkenyl, alkynyl, heterocycle, aryl, ORa, SRa, S(═O)Re, S(═O)₂Re, P(═O)₂Re, S(═O)₂ORe, P(═O)₂ORe, NRbRc, NRbS(═O)₂Re, NRbP(═O)₂Re, S(═O)₂NRbRc, P(═O)₂NRbRc. C(═O)ORd, C(═O)Ra, C(═O)NRbRc, OC(═O)Ra, OC(═O)NRbRc. NRbC(═O)ORe, NRdC(═O)NRbRc. NRdS(═O)₂NRbRc, NRdP(═O)₂NRbRc, NRbC(═O)Ra, or NRbP(═O)₂Re, wherein each occurrence of Ra is independently hydrogen, alkyl, cycloalkyl, alkenyl, cycloalkenyl, alkynyl, heterocycle, or aryl; each occurrence of Rb, Rc, and Rd is independently hydrogen, alkyl, cycloalkyl, heterocycle, aryl, or said Rb and Re together with the N to which they are bonded optionally form a heterocycle; and each occurrence of Re is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle, and aryl substituents can themselves be optionally substituted.

The term “cycloalkenyl” refers to a partially unsaturated cyclic hydrocarbon group containing 1 to 4 rings and 3 to 8 carbons per ring. Exemplary such groups include cyclobutenyl, cyclopentenyl, cyclohexenyl, etc. “Substituted cycloalkenyl” refers to a cycloalkenyl group substituted with one more substituents, preferably 1 to 4 substituents, at any available point of attachment. Exemplary substituents include but are not limited to one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORa, SRa, S(═O)Re, S(═O)₂Re, P(═O)₂Re, S(═O)₂ORe, P(═O)₂ORe, NRbRc. NRbS(═O)₂Re, NRbP(═O)₂Re, S(═O)₂NRbRc, P(═O)₂NRbRc. C(═O)ORd, C(═O)Ra, C(═O)NRbRc, OC(═O)Ra, OC(═O)NRbRc, NRbC(═O)ORe, NRdC(═O)NRbRc, NRdS(═O)₂NRbRc, NRdP(═O)₂NRbRc, NRbC(═O)Ra, or NRbP(═O)₂Re, wherein each occurrence of Ra is independently hydrogen, alkyl, cycloalkyl, alkenyl, cycloalkenyl, alkynyl, heterocycle, or aryl; each occurrence of Rb. Rc. and Rd is independently hydrogen, alkyl, cycloalkyl, heterocycle, aryl, or said Rb and Rc together with the N to which they are bonded optionally form a heterocycle; and each occurrence of Re is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle, and aryl substituents can themselves be optionally substituted.

The term “aryl” refers to cyclic, aromatic hydrocarbon groups that have 1 to 5 aromatic rings, especially monocyclic or bicyclic groups such as phenyl, biphenyl, or naphthyl. Where containing two or more aromatic rings (bicyclic, etc.), the aromatic rings of the aryl group may be joined at a single point (e.g., biphenyl), or fused (e.g., naphthyl, phenanthrenyl, and the like). “Substituted aryl” refers to an aryl group substituted by one or more substituents, preferably 1 to 3 substituents, at any available point of attachment. Exemplary substituents include but are not limited to one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORa, SRa. S(═O)Re, S(═O)₂Re. P(═O)₂Re, S(═O)₂ORe, P(═O)₂ORe. NRbRc, NRbS(═O)₂Re, NRbP(═O)₂Re, S(═O)₂NRbRc. P(═O)₂NRbRc, C(═O)ORd, C(═O)Ra, C(═O)NRbRc, OC(═O)Ra, OC(═O)NRbRc, NRbC(═O)ORe, NRdC(═O)NRbRc, NRdS(═O)₂NRbRc, NRdP(═O)₂NRbRc, NRbC(═O)Ra, or NRbP(═O)₂Re, wherein each occurrence of Ra is independently hydrogen, alkyl, cycloalkyl, alkenyl, cycloalkenyl, alkynyl, heterocycle, or aryl; each occurrence of Rb, Rc, and Rd is independently hydrogen, alkyl, cycloalkyl, heterocycle, aryl, or said Rb and Rc together with the N to which they are bonded optionally form a heterocycle; and each occurrence of Re is independently alkyl, cycloalkyl, alkenyl, cycloalkenyl, alkynyl, heterocycle, or aryl. The exemplary substituents can themselves be optionally substituted. Exemplary substituents also include fused cyclic groups, especially fused cycloalkyl, fused cycloalkenyl, fused heterocycle, or fused aryl, where the aforementioned cycloalkyl, cycloalkenyl, heterocycle, and aryl substituents can themselves be optionally substituted.

The term “carbocycle” refers to a fully saturated or partially saturated cyclic hydrocarbon group containing from 1 to 4 rings and 3 to 8 carbons per ring, or cyclic, aromatic hydrocarbon groups that have 1 to 5 aromatic rings, especially monocyclic or bicyclic groups such as phenyl, biphenyl, or naphthyl. The term “carbocycle” encompasses cycloalkyl, cycloalkenyl, cycloalkynyl, and aryl as defined hereinabove. The term “substituted carbocycle” refers to carbocycle or carbocyclic groups substituted with one or more substituents, preferably 1 to 4 substituents, at any available point of attachment. Exemplary substituents include, but are not limited to, those described above for substituted cycloalkyl, substituted cycloalkenyl, substituted cycloalkynyl, and substituted aryl. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle, and aryl substituents can themselves be optionally substituted.

The terms “heterocycle” and “heterocyclic” refer to fully saturated, or partially or fully unsaturated, including aromatic (i.e., “heteroaryl”) cyclic groups (for example, 4 to 7 membered monocyclic, 7 to 11 membered bicyclic, or 8 to 16 membered tricyclic ring systems) which have at least one heteroatom in at least one carbon atom-containing ring. Each ring of the heterocyclic group containing a heteroatom may have 1, 2, 3, or 4 heteroatoms selected from nitrogen atoms, oxygen atoms, and/or sulfur atoms, where the nitrogen and sulfur heteroatoms may optionally be oxidized and the nitrogen heteroatoms may optionally be quaternized. (The term “heteroarylium” refers to a heteroaryl group bearing a quaternary nitrogen atom and thus a positive charge.) The heterocyclic group may be attached to the remainder of the molecule at any heteroatom or carbon atom of the ring or ring system. Exemplary monocyclic heterocyclic groups include azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like. Exemplary bicyclic heterocyclic groups include indolyl, isoindolyl, benzothiazolyl, benzoxazolyl, benzoxadiazolyl, benzothienyl, benzo[d][1,3]dioxolyl, 2,3-dihydrobenzo[b][1,4]dioxinvl, quinuclidinyl, quinolinyl, tetrahydroisoquinolinyl, isoquinolinyl, benzimidazolyl, benzopyranyl, indolizinyl, benzofuryl, benzofurazanyl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl], or furo[2,3-b]pyridinyl), dihydroisoindolyl, dihydroquinazolinyl (such as 3,4-dihydro-4-oxo-quinazolinvl), triazinvlazepinyl, tetrahydroquinolinyl, and the like. Exemplary tricyclic heterocyclic groups include carbazolyl, benzidolyl, phenanthrolinyl, acridinyl, phenanthridinyl, xanthenyl, and the like.

“Substituted heterocycle” and “substituted heterocyclic” (such as “substituted heteroaryl”) refer to heterocycle or heterocyclic groups substituted with one or more substituents, preferably 1 to 4 substituents, at any available point of attachment. Exemplary substituents include but are not limited to one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORa, SRa, S(═O)Re, S(═O)₂Re, P(═O)₂Re, S(═O)₂ORe, P(═O)₂ORe, NRbRc, NRbS(═O)₂Re, NRbP(═O)₂Re, S(═O)₂NRbRc. P(═O)₂NRbRc, C(═O)ORd, C(═O)Ra, C(═O)NRbRc. OC(═O)Ra, OC(═O)NRbRc, NRbC(═O)ORe, NRdC(═O)NRbRc. NRdS(═O)₂NRbRc, NRdP(═O)₂NRbRc, NRbC(═O)Ra, or NRbP(═O)₂Re, wherein each occurrence of Ra is independently hydrogen, alkyl, cycloalkyl, alkenyl, cycloalkenyl, alkynyl, heterocycle, or aryl; each occurrence of Rb, Rc, and Rd is independently hydrogen, alkyl, cycloalkyl, heterocycle, aryl, or said Rb and Rc together with the N to which they are bonded optionally form a heterocycle; and each occurrence of Re is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle, and aryl substituents can themselves be optionally substituted.

The term “alkylamino” refers to a group having the structure —NHR′, wherein R′ is hydrogen, alkyl or substituted alkyl, or cycloalkyl or substituted cyclolalkyl, as defined herein. Examples of alkylamino groups include, but are not limited to, methylamino, ethylamino, n-propylamino, iso-propylamino, cyclopropylamino, n-butylamino, tert-butylamino, neopentylamino, n-pentylamino, hexylamino, cyclohexylamino, and the like.

The term “dialkylamino” refers to a group having the structure —NRR′ wherein R and R′ are each independently alkyl or substituted alkyl, cycloalkyl or substituted cycloalkyl, cycloalkenyl or substituted cyclolalkenyl, aryl or substituted aryl, or heterocylyl or substituted heterocyclyl, as defined herein. R and R′ may be the same or different in a dialkyamino moiety. Examples of dialkylamino groups include, but are not limited to, dimethylamino, methyl ethylamino, diethylamino, methylpropylamino, di(n-propyl)amino, di(iso-propyl)amino, di(cyclopropyl)amino, di(n-butyl)amino, di(tert-butyl)amino, di(neopentyl)amino, di(n-pentyl)amino, di(hexyl)amino, di(cyclohexyl)amino, and the like. In some embodiments. R and R′ are linked to form a cyclic structure. The resulting cyclic structure may be aromatic or non-aromatic. Examples of cyclic diaminoalkyl groups include, but are not limited to, aziridinyl, pyrrolidinyl, piperidinyl, morpholinyl, pyrrolyl, imidazolyl. 1,3,4-trianolyl, and tetrazolyl.

The terms “halogen” or “halo” refer to chlorine, bromine, fluorine, or iodine.

Unless otherwise indicated, any heteroatom with unsatisfied valences is assumed to have hydrogen atoms sufficient to satisfy the valences.

Compounds of the present disclosure, and salts or solvates thereof, in some embodiments, exist in their tautomeric form (for example, as an amide or imino ether). All such tautomeric forms are contemplated herein as part of the present disclosure.

All stereoisomers of the present compounds (for example, those which may exist due to asymmetric carbons on various substituents), including enantiomeric forms and diastereomeric forms, are contemplated within the scope of this disclosure. Individual stereoisomers of the compounds of the disclosure may, for example, be substantially free of other isomers (e.g., as a pure or substantially pure optical isomer having a specified activity), or may be admixed, for example, as racemates or with all other, or other selected, stereoisomers. The chiral centers of the present disclosure, in some embodiments, have the S or R configuration as defined by the International Union of Pure and Applied Chemistry (IUPAC) 1974 Recommendations. The racemic forms can be resolved by physical methods, such as, for example, fractional crystallization, separation or crystallization of diastereomeric derivatives, or separation by chiral column chromatography. The individual optical isomers can be obtained from the racemates by any suitable method, including without limitation, conventional methods, such as, for example, salt formation with an optically active acid followed by crystallization.

Compounds of the present disclosure are, subsequent to their preparation, preferably isolated and purified to obtain a composition containing an amount by weight equal to or greater than 90%, for example, equal to greater than 95%, equal to or greater than 99% of the compounds (“substantially pure” compounds), which is then used or formulated as described herein. Such “substantially pure” compounds of the present disclosure are also contemplated herein as part of the present disclosure.

All configurational isomers of the compounds of the present disclosure are contemplated, either in admixture or in pure or substantially pure form. The definition of compounds of the present disclosure embraces both cis (Z) and trans (E) alkene isomers, as well as cis and trans isomers of cyclic hydrocarbon or heterocyclic rings.

Definitions of specific functional groups and chemical terms are described in more detail below. For purposes of this disclosure, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in “Organic Chemistry,” Thomas Sorrell, University Science Books, Sausalito (1999), the entire contents of which are incorporated herein by reference.

Certain compounds of the present disclosure, in some embodiments, exist in particular geometric or stereoisomeric forms. The present disclosure contemplates all such compounds, including cis- and trans-isomers, R- and S-enantiomers, diastereomers, (d)-isomers, (1)-isomers, the racemic mixtures thereof, and other mixtures thereof, as falling within the scope of the disclosure. Additional asymmetric carbon atoms, in some embodiments, are present in a substituent such as an alkyl group. All such isomers, as well as mixtures thereof, are intended to be included in this disclosure.

Isomeric mixtures containing any of a variety of isomer ratios, in some embodiments, are utilized in accordance with the present disclosure. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present disclosure. Those of ordinary skill in the art will readily appreciate that analogous ratios are contemplated for more complex isomer mixtures.

The present disclosure also includes isotopically-labeled compounds, which are identical to the compounds disclosed herein, but for the fact that one or more atoms are replaced by an atom having an atomic mass or mass number different from the atomic mass or mass number usually found in nature. Examples of isotopes that can be incorporated into compounds of the present disclosure include isotopes of hydrogen, carbon, nitrogen, oxygen, phosphorous, sulfur, fluorine, and chlorine, such as 2H, 3H, 13C, 11C, 14C, 15N, 18O, 17O, 31P, 32P, 35S, 18F, and 36Cl, respectively. Compounds of the present disclosure, or an enantiomer, diastereomer, tautomer, or pharmaceutically acceptable salt or solvate thereof, which contain the aforementioned isotopes and/or other isotopes of other atoms are within the scope of this disclosure. Certain isotopically-labeled compounds of the present disclosure, for example those into which radioactive isotopes such as 3H and 14C are incorporated, are useful in drug and/or substrate tissue distribution assays. Tritiated, i.e., 3H, and carbon-14, i.e., 14C, isotopes are particularly preferred for their ease of preparation and detectability. Further, substitution with heavier isotopes such as deuterium. i.e., 2H, can afford certain therapeutic advantages resulting from greater metabolic stability, for example increased in vivo half-life or reduced dosage requirements and hence may be preferred in some circumstances. Isotopically labeled compounds can generally be prepared by carrying out the procedures disclosed in the Schemes and/or in the Examples below, by substituting a readily-available isotopically labeled reagent for a non-isotopically labeled reagent.

If, for instance, a particular enantiomer of a compound of the present disclosure is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically active acid or base, followed by resolution of the diastereomers thus formed by fractional crystallization or chromatographic means well known in the art, and subsequent recovery of the pure enantiomers.

It will be appreciated that the compounds, as described herein, in some embodiments, are substituted with any number of substituents or functional moieties. In general, the term “substituted” whether preceded by the term “optionally” or not, and substituents contained in formulas of this disclosure, refer to the replacement of hydrogen radicals in a given structure with the radical of a specified substituent. When more than one position in any given structure, in some embodiments, are substituted with more than one substituent selected from a specified group, the substituent is either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic, substituents of organic compounds. For purposes of this disclosure, heteroatoms such as nitrogen may have hydrogen substituents and/or any permissible substituents of organic compounds described herein which satisfy the valencies of the heteroatoms. Furthermore, this disclosure is not intended to be limited in any manner by the permissible substituents of organic compounds. Combinations of substituents and variables envisioned by this disclosure are preferably those that result in the formation of stable compounds useful in the treatment, for example, of infectious diseases or proliferative disorders. The term “stable,” as used herein, preferably refers to compounds which possess stability sufficient to allow manufacture and which maintain the integrity of the compound for a sufficient period of time to be detected and preferably for a sufficient period of time to be useful for the purposes detailed herein.

### Methods of Treatment

Liver cancer is one of the leading causes of cancer mortality worldwide, and the incidence and mortality rates of liver cancer have been increasing for decades. For example, in the United States, the 2-year survival is less than 50% and 5-year survival is only 10% in hepatocellular carcinoma (HCC), a type of primary liver cancer. Currently available treatments of unresectable HCC include sorafenib, lenvatinib, a combination of atezolizumab and bevacizumab, and a combination of tremelimumab and durvalumab and second-line therapies such as regorafenib, cabozantinib, ramucirumab, single agent pembrolizumab or nivolumab, and combination of nivolumab with ipilimumab. Despite the available therapies, the prognosis for patients with liver cancer such as HCC remains poor, with survival estimates of about 6-20 months following diagnosis. In addition, safety and tolerability of the available therapies remain significant issues. For example, treatment-related grade 3-5 adverse events occur in more than about 45% of patients receiving either a combination of atezolizumab and bevacizumab or sorafenib as first-line therapy, and include, for example, proteinuria, hypertension, worsening of liver function, and serious (and in some cases fatal) bleeding events. In addition, the combination of tremelimumab and durvalumab (Single Tremelimumab Regular Interval Durvalumab) in a phase 2 trial for unresectable hepatocellular carcinoma exhibited grade 3-4 adverse events 50.5% of patients. A combination of lenvatinib and pembrolizumab demonstrated grade 3-5 adverse events occurring in 62.5%, with most common immune-mediated toxicity being hypothyroidism, and the study noted treatment discontinuation of 18% patients for the combination. Thus, there is a need to develop new therapies to treat liver cancers with reduced adverse events and increased tolerability.

The etiology of liver cancers, for example HCC, includes viral (e.g., viral infections) and non-viral. Nonviral etiologies may be associated with certain conditions and/or exposure, such as but not limited to, alcoholic liver disease, alcoholic cirrhosis, autoimmune disease, metabolic disease, alcoholic steatohepatitis, non-alcoholic fatty liver disease, non-alcoholic steatohepatitis, diabetes, obesity, iron overload syndrome, autoimmune hepatitis, primary biliary cholangitis, crvptogenic liver disease, alcohol use, tobacco use, oral contraceptive use, pesticide exposure, aflatoxin exposure, or betel quid chewing. Effective and safe treatments for non-viral groups especially remain elusive with currently available treatments. For example, in the IMbrave150 study on the efficacy and safety for the combination of atezolizumab and bevacizumab, patients who had non-viral etiology of HCC had a hazard ratio for death of greater than 1 (1.05) and had no survival benefit (measured by progress-free survival or overall survival) over sorafenib.

The methods provided herein, in some embodiments, result in durable partial responses in patients with non-viral liver cancer. Furthermore, the methods of treatment provided herein, in some embodiments, may result in reduced or limited toxicities (e.g., diarrhea and nausea) that are generally manageable or do not overlap significantly with those of currently available treatment regimens. The methods provided herein thus provide opportunities for improving overall survival of patients with non-viral liver cancer, curbing adverse side effects, and maintaining tolerability when combined with other agents.

In some embodiments, disclosed herein are methods of treating a subject diagnosed with a non-viral liver cancer, the method comprising administering to the subject an effective amount of a compound of Formula (I):

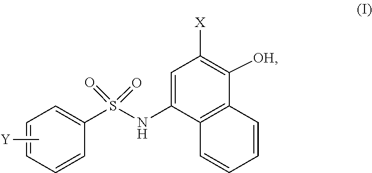

or a pharmaceutically acceptable salt thereof, wherein X is selected from the group consisting of hydrogen, phenylsulfanyl, hydroxy-naphthalenyl, quinolin-8-ylsulfanyl, triazol-3-yl sulfanyl, and benzothiazol-2-ylsulfanyl; and Y is selected from the group consisting of hydrogen, methyl, chloro, bromo, methoxy, ethoxy, tert-butyl, nitro, methyl ester, acetamide, 1,4 dioxine, fluoro, trifluoro methoxy, acetyl, trifluoro methyl, propyl, cyclohexene, methoxy-phenoxy, chloro phenoxy, tolyloxy, and phenoxy.

In some embodiments, the compound of Formula (I) is N-(1′,2-dihydroxy-1,2′-binaphthalen-4′-yl)-4-methoxybenzenesulfonamide. N-(3,1′-Dihydroxy-[1,2′]binaphthalenyl-4′-yl)-4-methoxy-benzenesulfonamide, N-(4,1′-Dihydroxy-[1,2′]binaphthalenyl-4′-yl)-4-methoxy-benzenesulfonamide, N-(5,1′-Dihydroxy-[1,2′]binaphthalenyl-4′-yl)-4-methoxy-benzenesulfonanide, N-(6,1′-Dihydroxy-[1,2′]binaphthalenyl-4′-yl)-4-methoxy-benzenesulfonamide, N-(7,1′-Dihydroxy-[1,2′]binaphthalenyl-4′-yl)-4-methoxy-benzenesulfonamide, N-(8,1′-Dihydroxy-[1,2′]binaphthalenyl-4′-yl)-4-methoxy-benzenesulfonamide, 4-Bromo-N-(1,6′-dihydroxy-[2,2′]binaphthalenyl-4-yl)-benzenesulfonamide, or 4-Bromo-N-[4-hydroxy-3-(1H-[1,2,4]triazol-3-ylsulfanyl)-naphthalen-1-yl]-benzenesulfonamide, or a pharmaceutically acceptable salt thereof.

In some embodiments, the compound of Formula (I) is N-(1′,2-dihydroxy-1,2′-binaphthalen-4′-yl)-4-methoxybenzenesulfonamide, having the following structure:

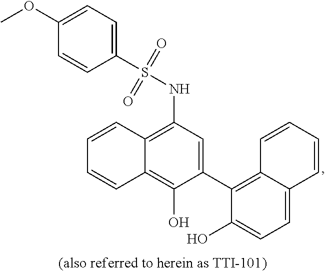 [SMILES: COc1ccc(S(=O)(=O)Nc2cc(-c3c(O)ccc4ccccc34)c(O)c3ccccc23)cc1]

or a pharmaceutically acceptable salt thereof.

In some embodiments, the non-viral liver cancer is hepatocellular carcinoma, fibrolamellar carcinoma, bile duct cancer, angiosarcoma, or hepatoblastoma. In some embodiments, the non-viral liver cancer is hepatocellular carcinoma.

In some embodiments, the subject has alcoholic liver disease, alcoholic cirrhosis, autoimmune disease, metabolic disease, alcoholic steatohepatitis, non-alcoholic fatty liver disease, non-alcoholic steatohepatitis, diabetes, obesity, iron overload syndrome, autoimmune hepatitis, primary biliary cholangitis, cryptogenic liver disease, alcohol use, tobacco use, oral contraceptive use, pesticide exposure, aflatoxin exposure, or betel quid chewing. In some embodiments, the subject has alcoholic steatohepatitis, alcoholic liver disease, non-alcoholic fatty liver disease, or non-alcoholic steatohepatitis. In some embodiments, the subject has alcoholic steatohepatitis. In some embodiments, the subject has non-alcoholic steatohepatitis.

In some embodiments, the subject is HBV negative. In some embodiments, the subject is negative for hepatitis B surface antigen (HBsAg). In some embodiments, the subject is negative for hepatitis B virus (HBV). In some embodiments, the subject is positive for hepatitis B surface antibody (HBsAb or anti-HBs) and negative for hepatitis B core antibody (HBcAb or anti-HBc). In some embodiments, the subject is positive for HBsAb and positive for HBcAb. In some embodiments, the subject is negative for HBsAg, negative for HBsAb, and negative for HBcAb. In some embodiments, the subject is negative for hepatitis B e-antigen (HBeAg). In some embodiments, the subject is negative for IgM anti-HBc. In some embodiments, the subject is negative for IgG anti-HBc.

In some embodiments, the subject is HCV negative. In some embodiments, the subject is negative for hepatitis C virus (HCV). In some embodiments, the subject is negative for hepatitis C antibody (HCVAb or anti-HCV). In some embodiments, the subject is positive for HCVAb and negative for HCV RNA (e.g., through nucleic acid test or PCT test).

In some embodiments, the subject has been administered one or more prior cancer therapy.

In some embodiments, the prior cancer therapy is sorafenib, pembrolizumab, nivolumab, lenvatinib, bevacizumab, atezolizumab, ramucirumab, cabozantinib, infigratinib, pemigatinib, regorafenib, tremelimumab, or durvalumab, or pharmaceutically acceptable salts thereof, or any combination thereof. In some embodiments, the prior cancer therapy is sorafenib, pembrolizumab, nivolumab, lenvatinib, bevacizumab, tremelimumab, or durvalumab, or any combination thereof. In some embodiments, the prior cancer therapy is sorafenib. In some embodiments, the prior cancer therapy is a combination of atezolizumab and bevacizumab. In some embodiments, the prior cancer therapy is lenvatinib. In some embodiments, the prior cancer therapy is a combination of tremelimumab and durvalumab.

In some embodiments, the non-viral liver cancer has progressed from, or the subject was intolerant to, a prior first line cancer therapy. In some embodiments, the first line cancer therapy is sorafenib. In some embodiments, the first line cancer therapy is a combination of atezolizumab and bevacizumab. In some embodiments, the first line cancer therapy is lenvatinib. In some embodiments, the first line cancer therapy is a combination of tremelimumab and durvalumab.

In some embodiments, non-viral liver cancer has progressed from, or the subject was intolerant to, a prior second line cancer therapy. In some embodiments, the second line cancer therapy is regorafenib, cabozantinib, ramucirumab, single agent pembrolizumab or nivolumab, or the combination of nivolumab with ipilimumab.

In some embodiments, non-viral liver cancer has progressed from, or the subject was intolerant to, a prior third line cancer therapy. In some embodiments, the third line cancer therapy is regorafenib, cabozantinib, ramucirumab, single agent pembrolizumab or nivolumab, or the combination of nivolumab with ipilimumab.

In some embodiments, the non-viral liver cancer is treatment naïve (has not been previously administered a cancer therapy).

In some embodiments, about 1 mg/kg/day to about 100 mg/kg/day of the compound of Formula (I), or a pharmaceutically acceptable salt thereof, is administered to the subject. In some embodiments, about 1 mg/kg/day to about 90 mg/kg/day of the compound of Formula (I), or a pharmaceutically acceptable salt thereof, is administered to the subject. In some embodiments, about 1 mg/kg/day to about 80 mg/kg/day of the compound of Formula (I), or a pharmaceutically acceptable salt thereof, is administered to the subject. In some embodiments, about 1 mg/kg/day to about 70 mg/kg/day of the compound of Formula (I), or a pharmaceutically acceptable salt thereof, is administered to the subject. In some embodiments, about 1 mg/kg/day to about 60 mg/kg/day of the compound of Formula (I), or a pharmaceutically acceptable salt thereof, is administered to the subject. In some embodiments, about 1 mg/kg/day to about 50 mg/kg/day of the compound of Formula (I), or a pharmaceutically acceptable salt thereof, is administered to the subject. In some embodiments, about 5 mg/kg/day to about 50 mg/kg/day of the compound of Formula (I), or a pharmaceutically acceptable salt thereof, is administered to the subject. In some embodiments, about 5 mg/kg/day to about 40 mg/kg/day of the compound of Formula (I), or a pharmaceutically acceptable salt thereof, is administered to the subject. In some embodiments, about 5 mg/kg/day to about 30 mg/kg/day of the compound of Formula (I), or a pharmaceutically acceptable salt thereof, is administered to the subject. In some embodiments, about 5 mg/kg/day to about 8 mg/kg/day of the compound of Formula (I), or a pharmaceutically acceptable salt thereof, is administered to the subject. In some embodiments, about 10 mg/kg/day to about 15 mg/kg/day of the compound of Formula (I), or a pharmaceutically acceptable salt thereof, is administered to the subject. In some embodiments, about 20 mg/kg/day to about 30 mg/kg/day of the compound of Formula (I), or a pharmaceutically acceptable salt thereof, is administered to the subject. In some embodiments, about 0.5 mg/kg/dose to about 25 mg/kg/dose of the compound of Formula (I), or a pharmaceutically acceptable salt thereof, is administered to the subject. In some embodiments, about 2.5 mg/kg/dose to about 15 mg/kg/dose of the compound of Formula (I), or a pharmaceutically acceptable salt thereof, is administered to the subject. In some embodiments, about 2.5 mg/kg/day to about 4 mg/kg/day of the compound of Formula (I), or a pharmaceutically acceptable salt thereof, is administered to the subject. In some embodiments, about 5 mg/kg/day to about 7.5 mg/kg/day of the compound of Formula (I), or a pharmaceutically acceptable salt thereof, is administered to the subject. In some embodiments, about 10 mg/kg/day to about 15 mg/kg/day of the compound of Formula (I), or a pharmaceutically acceptable salt thereof, is administered to the subject.

In some embodiments, the subject is dosed twice daily of the compound of Formula (I), or a pharmaceutically acceptable salt thereof. In some embodiments, the subject is dosed twice daily of the compound of Formula (I), or a pharmaceutically acceptable salt thereof, at about 1.6 mg/kg/dose. In some embodiments, the subject is dosed twice daily of the compound of Formula (I), or a pharmaceutically acceptable salt thereof, at about 3.2 mg/kg/dose. In some embodiments, the subject is dosed twice daily of the compound of Formula (I), or a pharmaceutically acceptable salt thereof, at about 6.4 mg/kg/dose. In some embodiments, the subject is dosed twice daily of the compound of Formula (I), or a pharmaceutically acceptable salt thereof, at about 12.8 mg/kg/dose.

In some embodiments, about 100 mg to about 5000 mg of the compound of Formula (I), or a pharmaceutically acceptable salt thereof, is administered to the subject per day. In some embodiments, about 200 mg to about 5000 mg of the compound of Formula (I), or a pharmaceutically acceptable salt thereof, is administered to the subject per day. In some embodiments, about 200 mg to about 4000 mg of the compound of Formula (I), or a pharmaceutically acceptable salt thereof, is administered to the subject per day. In some embodiments, about 200 mg to about 3000 mg of the compound of Formula (I), or a pharmaceutically acceptable salt thereof, is administered to the subject per day. In some embodiments, about 200 mg to about 2000 mg of the compound of Formula (I), or a pharmaceutically acceptable salt thereof, is administered to the subject per day. In some embodiments, about 200 mg to about 1000 mg of the compound of Formula (I), or a pharmaceutically acceptable salt thereof, is administered to the subject per day. In some embodiments, about 200 mg to about 800 mg of the compound of Formula (I), or a pharmaceutically acceptable salt thereof, is administered to the subject per day. In some embodiments, about 600 mg to about 5000 mg of the compound of Formula (I), or a pharmaceutically acceptable salt thereof, is administered to the subject per day. In some embodiments, about 600 mg to about 4000 mg of the compound of Formula (I), or a pharmaceutically acceptable salt thereof, is administered to the subject per day. In some embodiments, about 600 mg to about 3000 mg of the compound of Formula (I), or a pharmaceutically acceptable salt thereof, is administered to the subject per day. In some embodiments, about 600 mg to about 2000 mg of the compound of Formula (I), or a pharmaceutically acceptable salt thereof, is administered to the subject per day. In some embodiments, about 600 mg to about 1600 mg of the compound of Formula (I), or a pharmaceutically acceptable salt thereof, is administered to the subject per day. In some embodiments, about 600 mg to about 1400 mg of the compound of Formula (I), or a pharmaceutically acceptable salt thereof, is administered to the subject per day. In some embodiments, about 600 mg to about 1200 mg of the compound of Formula (I), or a pharmaceutically acceptable salt thereof, is administered to the subject per day. In some embodiments, about 600 mg to about 1000 mg of the compound of Formula (I), or a pharmaceutically acceptable salt thereof, is administered to the subject per day. In some embodiments, about 1000 mg to about 5000 mg of the compound of Formula (I), or a pharmaceutically acceptable salt thereof, is administered to the subject per day. In some embodiments, about 1000 mg to about 4000 mg of the compound of Formula (I), or a pharmaceutically acceptable salt thereof, is administered to the subject per day. In some embodiments, about 1000 mg to about 3000 mg of the compound of Formula (I), or a pharmaceutically acceptable salt thereof, is administered to the subject per day. In some embodiments, about 1000 mg to about 2000 mg of the compound of Formula (I), or a pharmaceutically acceptable salt thereof, is administered to the subject per day. In some embodiments, about 1000 mg to about 1600 mg of the compound of Formula (I), or a pharmaceutically acceptable salt thereof, is administered to the subject per day. In some embodiments, about 1400 mg to about 5000 mg of the compound of Formula (I), or a pharmaceutically acceptable salt thereof, is administered to the subject per day. In some embodiments, about 1400 mg to about 4000 mg of the compound of Formula (I), or a pharmaceutically acceptable salt thereof, is administered to the subject per day. In some embodiments, about 1400 mg to about 3000 mg of the compound of Formula (I), or a pharmaceutically acceptable salt thereof, is administered to the subject per day. In some embodiments, about 1400 mg to about 2000 mg of the compound of Formula (I), or a pharmaceutically acceptable salt thereof, is administered to the subject per day.

In some embodiments, about 400 mg to about 1600 mg of the compound of Formula (I), or a pharmaceutically acceptable salt thereof, is administered to the subject per day. In some embodiments, about 400 mg to about 800 mg of the compound of Formula (I), or a pharmaceutically acceptable salt thereof, is administered to the subject per day. In some embodiments, about 800 mg to about 1200 mg of the compound of Formula (I), or a pharmaceutically acceptable salt thereof, is administered to the subject per day. In some embodiments, about 1200 mg to about 1600 mg of the compound of Formula (I), or a pharmaceutically acceptable salt thereof, is administered to the subject per day.

In some embodiments, about 100 mg of the compound of Formula (I), or a pharmaceutically acceptable salt thereof, is administered to the subject per day. In some embodiments, about 200 mg of the compound of Formula (I), or a pharmaceutically acceptable salt thereof, is administered to the subject per day. In some embodiments, about 300 mg of the compound of Formula (I), or a pharmaceutically acceptable salt thereof, is administered to the subject per day. In some embodiments, about 400 mg of the compound of Formula (I), or a pharmaceutically acceptable salt thereof, is administered to the subject per day. In some embodiments, about 500 mg of the compound of Formula (I), or a pharmaceutically acceptable salt thereof, is administered to the subject per day. In some embodiments, about 600 mg of the compound of Formula (I), or a pharmaceutically acceptable salt thereof, is administered to the subject per day. In some embodiments, about 700 mg of the compound of Formula (I), or a pharmaceutically acceptable salt thereof, is administered to the subject per day. In some embodiments, about 800 mg of the compound of Formula (I), or a pharmaceutically acceptable salt thereof, is administered to the subject per day. In some embodiments, about 900 mg of the compound of Formula (I), or a pharmaceutically acceptable salt thereof, is administered to the subject per day. In some embodiments, about 1000 mg of the compound of Formula (I), or a pharmaceutically acceptable salt thereof, is administered to the subject per day. In some embodiments, about 1100 mg of the compound of Formula (I), or a pharmaceutically acceptable salt thereof, is administered to the subject per day. In some embodiments, about 1200 mg of the compound of Formula (I), or a pharmaceutically acceptable salt thereof, is administered to the subject per day. In some embodiments, about 1300 mg of the compound of Formula (I), or a pharmaceutically acceptable salt thereof, is administered to the subject per day. In some embodiments, about 1400 mg of the compound of Formula (I), or a pharmaceutically acceptable salt thereof, is administered to the subject per day. In some embodiments, about 1500 mg of the compound of Formula (I), or a pharmaceutically acceptable salt thereof, is administered to the subject per day. In some embodiments, about 1600 mg of the compound of Formula (I), or a pharmaceutically acceptable salt thereof, is administered to the subject per day. In some embodiments, about 1700 mg of the compound of Formula (I), or a pharmaceutically acceptable salt thereof, is administered to the subject per day. In some embodiments, about 1800 mg of the compound of Formula (I), or a pharmaceutically acceptable salt thereof, is administered to the subject per day. In some embodiments, about 1900 mg of the compound of Formula (I), or a pharmaceutically acceptable salt thereof, is administered to the subject per day. In some embodiments, about 2000 mg of the compound of Formula (I), or a pharmaceutically acceptable salt thereof, is administered to the subject per day. In some embodiments, about 2200 mg of the compound of Formula (I), or a pharmaceutically acceptable salt thereof, is administered to the subject per day. In some embodiments, about 2400 mg of the compound of Formula (I), or a pharmaceutically acceptable salt thereof, is administered to the subject per day. In some embodiments, about 2600 mg of the compound of Formula (I), or a pharmaceutically acceptable salt thereof, is administered to the subject per day. In some embodiments, about 2800 mg of the compound of Formula (I), or a pharmaceutically acceptable salt thereof, is administered to the subject per day. In some embodiments, about 3000 mg of the compound of Formula (I), or a pharmaceutically acceptable salt thereof, is administered to the subject per day.

In some embodiments, the method further comprises administering to the subject one or more additional agent.

In some embodiments, the additional agent is selected from the group consisting of an angiogenesis inhibitor, an anti-PD-1 antibody, and an anti-PD-L1 antibody.

In some embodiments, the additional agent is an anti-PD-1 antibody, wherein the anti-PD-1 antibody is cemiplimab, nivolumab, pembrolizumab, pidilizumab, spartalizumab, camrelizumab, sintilimab, tislelizumab, toripalimab, dostarlimab, sasanlimab, retifanlimab, tebotelimab, ABBV-181, AK104, AK105, BCD-100, BI-754091, CBT-501, CC-90006, GLS-010, HLX10, IBI-308, JNJ-3283, JS001, LZM009, MEDI0680 (AMP-514), REGN-2810. SHR-1210, Sym021, TSR-042, or XmAb20717. In some embodiments, the anti-PD-1 antibody is pembrolizumab.

In some embodiments, the additional agent is an anti-PD-L1 antibody, wherein the anti-PD-L1 antibody is atezolizumab, avelumab, durvalumab, envafolimab, FS118, BCD-135, BGB-A333, BGBA-317, CBT-502, CK-301, CS1001, FAZ053, MDX-1105, MSB2311, SHR-1316. M7824, LY3415244, CA-170, or CX-072. In some embodiments, the anti-PD-L1 antibody is atezolizumab.

In some embodiments, the additional agent is an angiogenesis inhibitor, wherein the angiogenesis inhibitor is bevacizumab, sorafenib, sunitinib, nilotinib, pazopanib, dasatinib, regorafenib, cabozantinib, lenvatinib, ponatinib, ziv-aflibercept, axitinib, tivozanib, everolimus, lenalidomide, thalidomide, vandetanib, orvandetanib, or ramucirumab. In some embodiments, the angiogenesis inhibitor is bevacizumab.

In some embodiments, disclosed herein is a method of treating a subject diagnosed with a non-viral liver cancer, the method comprising administering to the subject an effective amount of a compound of Formula (II):

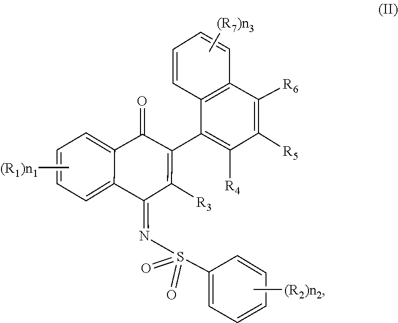

or a pharmaceutically acceptable salt thereof, wherein
   each occurrence of R₁ is independently hydrogen, halogen, cyano, nitro, CF₃, OCF₃, ORa, SRa, C(═O)Ra, OC(═O)Ra, C(═O)OR, NRbRc, NRbC(═O)Rc, C(═O)NRbRc, NRbC(═O)ORc, OC(═O)NRbRc, NRaC(═O)NRbRc, alkyl, alkenyl, cycloalkyl, optionally substituted aryl, or optionally substituted heterocycle:
n₁ is 0, 1, 2, 3, or 4;
each occurrence of R₂ is independently hydrogen, halogen, cyano, nitro, CF₃, OCF₃, ORa, SRa, C(═O)Ra. OC(═O)Ra, C(═O)ORa, NRbRc, NRbC(═O)Rc, C(═O)NRbRc, NRbC(═O)ORc, OC(═O)NRbRc, NRaC(═O)NRbRc, alkyl, alkenyl, cycloalkyl, cycloalkenyl, optionally substituted aryl, optionally substituted aryloxyl, or optionally substituted heterocycle;
n₂ is 0, 1, 2, 3, 4, or 5;
R₃ is hydrogen, halogen, cyano, nitro, CF₃, OCF₃, ORa, SRa, OC(═O)Ra, alkyl, alkenyl, cycloalkyl, or optionally substituted aryl or heteroaryl;
R₄ is hydrogen, halogen, cyano, nitro, CF₃, OCF₃, ORa, SRa, NRbRc, OC(═O)Ra, alkyl, alkenyl, or cycloalkyl;
each occurrence of R₅, R₆, and R₇ is independently hydrogen, halogen, cyano, nitro, CF₃, OCF₃, ORa, SRa, C(═O)Ra, OC(═O)Ra. C(═O)ORa, NRbRc. NRbC(═O)Rc, C(═O)NRbRc, NRbC(═O)ORc, OC(═O)NRbRc, NRaC(═O)NRbRc, alkyl, alkenyl, cycloalkyl, optionally substituted aryl, or optionally substituted heterocycle;
n₃ is 0, 1, 2, 3, or 4; and
each occurrence of Ra. Rb, and Rc is independently hydrogen, alkyl, cycloalkyl, alkenyl, cycloalkenyl, alkynyl, heterocycle, or aryl; or said Rb and Rc together with the nitrogen atom to which they are bonded optionally form a heterocycle comprising 1-4 heteroatoms.

In some embodiments of Formula (II), each occurrence of R₁ is independently hydrogen, halogen, cyano, nitro, CF₃, OCF₃, ORa, or SRa. In some embodiments of Formula (II), each occurrence of R₁ is independently C(═O)Ra, OC(═O)Ra, C(═O)ORa. NRaRb, NRbC(═O)Ra, C(═O)NRbRc, NRbC(═O)ORa, OC(═O)NRbRc, or NRaC(═O)NRbRc. In some embodiments of Formula (II), each occurrence of R₁ is independently alkyl, alkenyl, cycloalkyl, optionally substituted aryl, or optionally substituted heterocycle. In some embodiments of Formula (II), R₁ is hydrogen.

In some embodiments of Formula (II), n₁ is 0, 1, or 2. In some embodiments of Formula (II), n₁ is 1. In some embodiments of Formula (II), n₁ is 0.

In some embodiments of Formula (II), each occurrence of R₂ is independently hydrogen, halogen, cyano, nitro, CF₃, OCF₃, ORa, or SRa. In some embodiments of Formula (II), each occurrence of R₂ is independently C(═O)Ra, OC(═O)Ra, C(═O)ORa. NRaRb, NRbC(═O)Ra, C(═O)NRbRc, NRbC(═O)ORa, OC(═O)NRbRc, or NRaC(═O)NRbRc. In some embodiments of Formula (II), each occurrence of R₂ is independently alkyl, alkenyl, cycloalkyl, optionally substituted aryl, or optionally substituted heterocycle. In some embodiments of Formula (II), R₂ is hydrogen.

In some embodiments of Formula (II), n₂ is 0, 1, or 2. In some embodiments of Formula (II), n₂ is 1. In some embodiments of Formula (II), n₂ is 0.

In some embodiments of Formula (II), R₃ is hydrogen, halogen, cyano, nitro, or CF₃. In some embodiments of Formula (II). R₃ is OCF₃, ORa, SRa, or OC(═O)Ra. In some embodiments of Formula (II), R₃ is alkyl, alkenyl, or cycloalkyl. In some embodiments of Formula (II), R₃ is hydrogen.

In some embodiments of Formula (II), R₄ is hydrogen, halogen, cyano, nitro, or ORa. In some embodiments of Formula (II), R₄ is OCF₃, SRa, or OC(═O)Ra. In some embodiments of Formula (II), R₄ is alkyl, alkenyl, or cycloalkyl. In some embodiments of Formula (II), R₄ is OH. In some embodiments of Formula (II), R₄ is OMe.

In some embodiments of Formula (II), R₅, R₆, and R₇ are each independently selected from the group consisting of hydrogen, halogen, cyano, nitro, and CF₃. In some embodiments of Formula (II), R₅, R₆, and R₇ are each independently selected from the group consisting of OCF₃, ORa, and SRa. In some embodiments of Formula (II), R₅, R₆, and R₇ are each independently selected from the group consisting of OCF₃ and ORa. In some embodiments of Formula (II), R₅, R₆, and R₇ are each independently selected from the group consisting of C(═O)Ra, OC(═O)Ra, C(═O)ORa. NRaRb, NRbC(═O)Ra, C(═O)NRbRc, NRbC(═O)ORa, OC(═O)NRbRc, and NRaC(═O)NRbRc. In some embodiments of Formula (II), R₅, R₆, and R₇ are each independently selected from the group consisting of alkyl, alkenyl, cycloalkyl, optionally substituted aryl, and optionally substituted heterocycle. In some embodiments of Formula (II), each occurrence of R₅, R₆, and R₇ is hydrogen.

In some embodiments of Formula (II), n₃ is 0, 1, or 2. In some embodiments of Formula (II), n₃ is 1. In some embodiments of Formula (II), n₃ is 0.

In some embodiments of Formula (II), each occurrence of Ra is independently hydrogen, alkyl, heterocycle, or aryl. In some embodiments of Formula (II), each occurrence of Ra is independently hydrogen or alkyl. In some embodiments of Formula (II), each occurrence of Rb and Rc is independently hydrogen, alkyl, heterocycle, or aryl. In some embodiments of Formula (II), each occurrence of Rb and Rc is independently hydrogen or alkyl. In some embodiments of Formula (II), Rb and Rc together with the nitrogen atom to which they are bonded optionally form a heterocycle comprising 1-4 heteroatoms each selected from the group consisting of N, O, and S.

In some embodiments, the compound of Formula (II) is a compound of Formula (III):

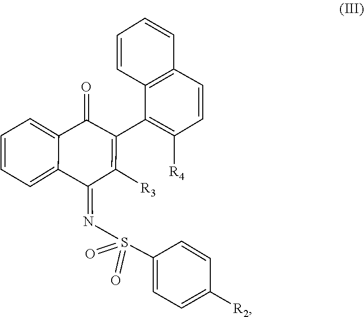

or a pharmaceutically acceptable salt thereof.

In some embodiments of Formula (III), R₂ is H, OH, alkyl, alkoxy, halogen, NRbRc, CF₃, OCF₃, or CN. In some embodiments of Formula (III), R₂ is NH₂, OH, OMe, OEt, OCH₂CH₂CH₃, or OCH(CH₃)₂. In some embodiments of Formula (III), R₂ is selected from the group consisting of hydrogen, methyl, ethyl, propyl, tert-butyl, F, Cl, Br, CF₃, nitro, methoxy, ethoxy, OCF₃, —C(═O)Me. —C(═O)OMe. —NHC(═O)Me. 1,4-dioxanyl, cyclohexanyl, cyclohexenyl, phenoxy, 2-methoxyphenoxy, 3-methoxyphenoxy, 4-methoxyphenoxy, 2-chlorophenoxy, 3-chlorophenoxy, 4-chlorophenoxy, 2-methylphenoxy, 3-methylphenoxy, and 4-methylphenoxy. In some embodiments of Formula (III), R₂ is OMe.

In some embodiments of Formula (III), R₃ is H, OH, alkyl, alkoxy, or halogen. In some embodiments of Formula (III), R₃ is H.

In some embodiments of Formula (III), R₄ is H, alkyl, OH, NH₂, alkoxy, halogen, CF₃, or CN. In some embodiments of Formula (III), R₄ is H, OH, or alkoxy. In some embodiments of Formula (III), R₄ is OH. In some embodiments of Formula (III), R₄ is OMe.

In some embodiments, the compound of Formula (III) is a compound of Formula (IV):

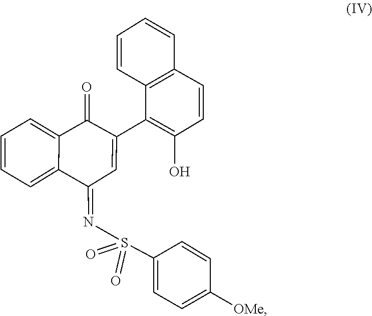 [SMILES: COc1ccc(S(=O)(=O)/N=C2\C=C(c3c(O)ccc4ccccc34)C(=O)c3ccccc32)cc1]

or a pharmaceutically acceptable salt thereof.

In some embodiments, the compound of Formula (II) is a compound selected from Examples of compounds shown in Table A, or a pharmaceutically acceptable salt thereof.

TABLE A
Selected compounds of Formula (II), where n₁, n₂, and n₃ are independently 1 or 2.

| Example | | | | | | | |
|---|---|---|---|---|---|---|---|
| No. | R₁ | R₂ | R₃ | R₄ | R₅ | R₆ | R₇ |
| 100 | H | Cl | H | H | H | F | F |
| 102 | F | H | F | H | F | OH | Cl |
| 103 | Cl | 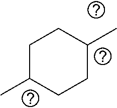 | Cl | OH | Cl | OH | NO₂ |
| 104 | CN | OH | CN | OH | Br | OMe | OC [symbol] |
| 105 | NO₂ | OMe | CF [symbol] | OH | NO₂ | NH₂ | SH |
| 106 | CFs | OEt | Me | OMe | OCF [symbol] | NH₂ | SH |
| 107 | OC [symbol] | OPr | Et | NH₂ | OCF [symbol] | SCH [symbol] | OH |
| 108 | OH | OBu | Pr | NH₂ | SH | COOH | CONH₂ |
| 109 | OH | NH₂ | Bu | NH₂ | SH | COOH | CONH₂ |
| 110 | SH | SH | Cyclopropyl | SH | OH | Ph | OH |
| 111 | COOH | Me | H | SCH₃ | OH | pyridinyl | OH |
| 112 | COOMe | CONH₂ | H | COOH | CONH₂ | Ph | OH |
| 113 | CONH₂ | NH(C═O)Me | —CH═CH₂ | COOH | CONH₂ | Me | H |
| 114 | CONME₂ | cyclopropyl | Ph | OH | CONH₂ | Et | H |
| 115 | NH [symbol] C═O)Me | Ph | pyridinyl | OH | NH(C═O) | Pr | Me |
| | | | | | Me | | |
| 116 | Ms | 3- | Me | OH | Me | cyclobutyl | Et |
| | | fluorophenyl | | | | | |
| 117 | Ph | 4- | H | NHMe | Et | CF₃ | cyclopropyl |
| | | pyridinyl | | | | | |
| 118 | 4-chlorophenyl | NO₂ | H | NHMe | cyclobutyl | OCF₃ | NO₂ |
| 119 | 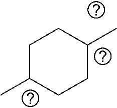 | CF₂ | H | NMe₂ | 4-chlorophenyl | SH | pyridinyl |
| 120 | cyclobutyl | OH | H | 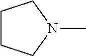 | Ph | Ph | 4-pyridinyl |
| 121 | 4-pyridinyl | SH | H | 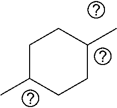 | OEt | 4-chlorophenyl | CONMe₂ |
| 122 | OEt | Me. | Me | Me | OPr | OPr | NH(C═O)Me |
| 123 | OPr | Et | Ph | Me | 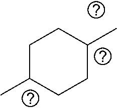 | 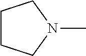 | 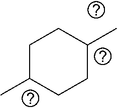 |
| 124 | OBu | Pr | Ph | Et | 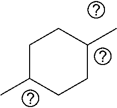 | 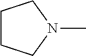 | 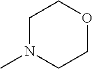 |

[symbol]

indicates data missing or illegible when filed

In some embodiments, the compound of Formula (III) is a compound selected from Examples of compounds shown in Table B, or a pharmaceutically acceptable salt thereof.

TABLE B
Selected compounds of Formula (III).

| Example No. | R₂ | R₃ | R₄ |
|---|---|---|---|
| 125 | Cl | H | H |
| 126 | H | F | OH |
| 127 | 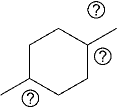 | Cl | OH |
| 128 | OH | H | OH |
| 129 | OMe | CF₃ | OH |
| 130 | OEt | Me | OMe |
| 131 | OPr | Et | NH₂ |
| 132 | [symbol] | Pr | NH₂ |
| 133 | NH₂ | Bu | NH₂ |
| 134 | SH | cyclopropyl | SH |
| 135 | Me | H | SCH [symbol] |
| 136 | CONH₂ | H | COOH |
| 137 | NH(C═O)Me | —CH═CH₂ | COOH |
| 138 | cyclopropyl | Ph | OH |
| 139 | Ph | pyridinyl | OH |
| 140 | 3-fluorophenyl | Me | OH |
| 141 | 4-pyridinyl | H | NHMe |
| 142 | NO₂ | H | NHMe |
| 143 | CF₃ | H | NMe₂ |
| 144 | OH | H | 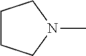 |
| 145 | SH | H | 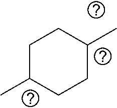 |
| 146 | Et | Ph | Me |
| 147 | Et | Ph | Me |
| 148 | Ph | Ph | Et |

[symbol]

indicates data missing or illegible when filed

In some embodiments, the non-viral liver cancer is hepatocellular carcinoma, fibrolamnellar carcinoma, bile duct cancer, angiosarcoma, or hepatoblastoma. In some embodiments, the non-viral liver cancer is hepatocellular carcinoma.

In some embodiments, the subject has alcoholic liver disease, alcoholic cirrhosis, autoimmune disease, metabolic disease, alcoholic steatohepatitis, non-alcoholic fatty liver disease, non-alcoholic steatohepatitis, diabetes, obesity, iron overload syndrome, autoimmune hepatitis, primary biliary cholangitis, cryptogenic liver disease, alcohol use, tobacco use, oral contraceptive use, pesticide exposure, aflatoxin exposure, or betel quid chewing. In some embodiments, the subject has alcoholic steatohepatitis, alcoholic liver disease, non-alcoholic fatty liver disease, or non-alcoholic steatohepatitis. In some embodiments, the subject has alcoholic steatohepatitis. In some embodiments, the subject has non-alcoholic steatohepatitis.

In some embodiments, the subject is HBV negative. In some embodiments, the subject is negative for hepatitis B surface antigen (HBsAg). In some embodiments, the subject is negative for hepatitis B virus (HBV). In some embodiments, the subject is positive for hepatitis B surface antibody (HBsAb or anti-HBs) and negative for hepatitis B core antibody (HBcAb or anti-HBc). In some embodiments, the subject is positive for HBsAb and positive for HBcAb. In some embodiments, the subject is negative for HBsAg, negative for HBsAb, and negative for HBcAb. In some embodiments, the subject is negative for hepatitis B e-antigen (HBeAg). In some embodiments, the subject is negative for IgM anti-HBc. In some embodiments, the subject is negative for IgG anti-HBc.

In some embodiments, the subject is HCV negative. In some embodiments, the subject is negative for hepatitis C virus (HCV). In some embodiments, the subject is negative for hepatitis C antibody (HCVAb or anti-HCV). In some embodiments, the subject is positive for HCVAb and negative for HCV RNA (e.g., through nucleic acid test or PCT test).

In some embodiments, the subject has been administered one or more prior cancer therapy.

In some embodiments, the prior cancer therapy is sorafenib, pembrolizumab, nivolumab, lenvatinib, bevacizumab, atezolizumab, ramucirumab, cabozantinib, infigratinib, pemigatinib, regorafenib, tremelimumab, or durvalumab, or pharmaceutically acceptable salts thereof, or any combination thereof. In some embodiments, the prior cancer therapy is sorafenib, pembrolizumab, nivolumab, lenvatinib, bevacizumab, tremelimumab, or durvalumab, or any combination thereof. In some embodiments, the prior cancer therapy is sorafenib. In some embodiments, the prior cancer therapy is a combination of atezolizumab and bevacizumab. In some embodiments, the prior cancer therapy is lenvatinib. In some embodiments, the prior cancer therapy is a combination of tremelimumab and durvalumab.

In some embodiments, the non-viral liver cancer has progressed from, or the subject was intolerant to, a prior first line cancer therapy. In some embodiments, the first line cancer therapy is sorafenib. In some embodiments, the first line cancer therapy is a combination of atezolizumab and bevacizumab. In some embodiments, the first line cancer therapy is lenvatinib. In some embodiments, the first line cancer therapy is a combination of tremelimumab and durvalumab.

In some embodiments, non-viral liver cancer has progressed from, or the subject was intolerant to, a prior second line cancer therapy. In some embodiments, the second line cancer therapy is regorafenib, cabozantinib, ramucirumab, single agent pembrolizumab or nivolumab, or the combination of nivolumab with ipilimumab.

In some embodiments, non-viral liver cancer has progressed from, or the subject was intolerant to, a prior third line cancer therapy.

In some embodiments, the non-viral liver cancer is treatment naïve (has not been previously administered a cancer therapy).

In some embodiments, about 1 mg/kg/day to about 100 mg/kg/day of the compound of Formula (II), or a pharmaceutically acceptable salt thereof, is administered to the subject. In some embodiments, about 1 mg/kg/day to about 90 mg/kg/day of the compound of Formula (II), or a pharmaceutically acceptable salt thereof, is administered to the subject. In some embodiments, about 1 mg/kg/day to about 80 mg/kg/day of the compound of Formula (II), or a pharmaceutically acceptable salt thereof, is administered to the subject. In some embodiments, about 1 mg/kg/day to about 70 mg/kg/day of the compound of Formula (II), or a pharmaceutically acceptable salt thereof, is administered to the subject. In some embodiments, about 1 mg/kg/day to about 60 mg/kg/day of the compound of Formula (II), or a pharmaceutically acceptable salt thereof, is administered to the subject. In some embodiments, about 1 mg/kg/day to about 50 mg/kg/day of the compound of Formula (II), or a pharmaceutically acceptable salt thereof, is administered to the subject. In some embodiments, about 5 mg/kg/day to about 50 mg/kg/day of the compound of Formula (II), or a pharmaceutically acceptable salt thereof, is administered to the subject. In some embodiments, about 5 mg/kg/day to about 40 mg/kg/day of the compound of Formula (II), or a pharmaceutically acceptable salt thereof, is administered to the subject. In some embodiments, about 5 mg/kg/day to about 30 mg/kg/day of the compound of Formula (II), or a pharmaceutically acceptable salt thereof, is administered to the subject. In some embodiments, about 5 mg/kg/day to about 8 mg/kg/day of the compound of Formula (II), or a pharmaceutically acceptable salt thereof, is administered to the subject. In some embodiments, about 10 mg/kg/day to about 15 mg/kg/day of the compound of Formula (II), or a pharmaceutically acceptable salt thereof, is administered to the subject. In some embodiments, about 20 mg/kg/day to about 30 mg/kg/day of the compound of Formula (II), or a pharmaceutically acceptable salt thereof, is administered to the subject. In some embodiments, about 0.5 mg/kg/dose to about 25 mg/kg/dose of the compound of Formula (II), or a pharmaceutically acceptable salt thereof, is administered to the subject. In some embodiments, about 2.5 mg/kg/dose to about 15 mg/kg/dose of the compound of Formula (II), or a pharmaceutically acceptable salt thereof, is administered to the subject. In some embodiments, about 2.5 mg/kg/day to about 4 mg/kg/day of the compound of Formula (II), or a pharmaceutically acceptable salt thereof, is administered to the subject. In some embodiments, about 5 mg/kg/day to about 7.5 mg/kg/day of the compound of Formula (II), or a pharmaceutically acceptable salt thereof, is administered to the subject. In some embodiments, about 10 mg/kg/day to about 15 mg/kg/day of the compound of Formula (II), or a pharmaceutically acceptable salt thereof, is administered to the subject.

In some embodiments, the subject is dosed twice daily of the compound of Formula (II), or a pharmaceutically acceptable salt thereof. In some embodiments, the subject is dosed twice daily of the compound of Formula (II), or a pharmaceutically acceptable salt thereof, at about 1.6 mg/kg/dose. In some embodiments, the subject is dosed twice daily of the compound of Formula (II), or a pharmaceutically acceptable salt thereof, at about 3.2 mg/kg/dose. In some embodiments, the subject is dosed twice daily of the compound of Formula (II), or a pharmaceutically acceptable salt thereof, at about 6.4 mg/kg/dose. In some embodiments, the subject is dosed twice daily of the compound of Formula (II), or a pharmaceutically acceptable salt thereof, at about 12.8 mg/kg/dose.

In some embodiments, about 100 mg of the compound of Formula (II), or a pharmaceutically acceptable salt thereof, is administered to the subject per day. In some embodiments, about 200 mg of the compound of Formula (II), or a pharmaceutically acceptable salt thereof, is administered to the subject per day. In some embodiments, about 300 mg of the compound of Formula (II), or a pharmaceutically acceptable salt thereof, is administered to the subject per day. In some embodiments, about 400 mg of the compound of Formula (II), or a pharmaceutically acceptable salt thereof, is administered to the subject per day. In some embodiments, about 500 mg of the compound of Formula (II), or a pharmaceutically acceptable salt thereof, is administered to the subject per day. In some embodiments, about 600 mg of the compound of Formula (II), or a pharmaceutically acceptable salt thereof, is administered to the subject per day. In some embodiments, about 700 mg of the compound of Formula (II), or a pharmaceutically acceptable salt thereof, is administered to the subject per day. In some embodiments, about 800 mg of the compound of Formula (II), or a pharmaceutically acceptable salt thereof, is administered to the subject per day. In some embodiments, about 900 mg of the compound of Formula (II), or a pharmaceutically acceptable salt thereof, is administered to the subject per day. In some embodiments, about 1000 mg of the compound of Formula (II), or a pharmaceutically acceptable salt thereof, is administered to the subject per day. In some embodiments, about 1100 mg of the compound of Formula (II), or a pharmaceutically acceptable salt thereof, is administered to the subject per day. In some embodiments, about 1200 mg of the compound of Formula (II), or a pharmaceutically acceptable salt thereof, is administered to the subject per day. In some embodiments, about 1300 mg of the compound of Formula (II), or a pharmaceutically acceptable salt thereof, is administered to the subject per day. In some embodiments, about 1400 mg of the compound of Formula (II), or a pharmaceutically acceptable salt thereof, is administered to the subject per day. In some embodiments, about 1500 mg of the compound of Formula (II), or a pharmaceutically acceptable salt thereof, is administered to the subject per day. In some embodiments, about 1600 mg of the compound of Formula (II), or a pharmaceutically acceptable salt thereof, is administered to the subject per day. In some embodiments, about 1700 mg of the compound of Formula (II), or a pharmaceutically acceptable salt thereof, is administered to the subject per day. In some embodiments, about 1800 mg of the compound of Formula (II), or a pharmaceutically acceptable salt thereof, is administered to the subject per day. In some embodiments, about 1900 mg of the compound of Formula (II), or a pharmaceutically acceptable salt thereof, is administered to the subject per day. In some embodiments, about 2000 mg of the compound of Formula (II), or a pharmaceutically acceptable salt thereof, is administered to the subject per day. In some embodiments, about 2200 mg of the compound of Formula (II), or a pharmaceutically acceptable salt thereof, is administered to the subject per day. In some embodiments, about 2400 mg of the compound of Formula (II), or a pharmaceutically acceptable salt thereof, is administered to the subject per day. In some embodiments, about 2600 mg of the compound of Formula (II), or a pharmaceutically acceptable salt thereof, is administered to the subject per day. In some embodiments, about 2800 mg of the compound of Formula (II), or a pharmaceutically acceptable salt thereof, is administered to the subject per day. In some embodiments, about 3000 mg of the compound of Formula (II), or a pharmaceutically acceptable salt thereof, is administered to the subject per day.

In some embodiments, the method further comprises administering to the subject one or more additional agent.

In some embodiments, the additional agent is selected from the group consisting of angiogenesis inhibitor, an anti-PD-1 antibody, and an anti-PD-L1 antibody.

In some embodiments, the additional agent is an anti-PD-1 antibody, wherein the anti-PD-1 antibody is cemiplimab, nivolumab, pembrolizumab, pidilizumab, spartalizumab, camrelizumab, sintilimab, tislelizumab, toripalimab, dostarlimab, sasanlimab, retifanlimab, tebotelimab, ABBV-181, AK104, AK105, BCD-100, BI-754091, CBT-501, CC-90006, GLS-010, HLX10, IBI-308, JNJ-3283, JS001. LZM009, MEDI0680 (AMP-514), REGN-2810. SHR-1210, Sym021, TSR-042, or XmAb20717. In some embodiments, the anti-PD-1 antibody is pembrolizumab.

In some embodiments, the additional agent is an anti-PD-L1 antibody, wherein the anti-PD-L1 antibody is atezolizumab, avelumab, durvalumab, envafolimab. FS118. BCD-135, BGB-A333, BGBA-317, CBT-502, CK-301, CS1001, FAZ053, MDX-1105. MSB2311. SHR-1316, M7824, LY3415244, CA-170, or CX-072. In some embodiments, the anti-PD-L1 antibody is atezolizumab.

In some embodiments, the additional agent is an angiogenesis inhibitor, wherein the angiogenesis inhibitor is bevacizumab, sorafenib, sunitinib, nilotinib, pazopanib, dasatinib, regorafenib, cabozantinib, lenvatinib, ponatinib, ziv-aflibercept, axitinib, tivozanib, everolimus, lenalidomide, thalidomide, vandetanib, orvandetanib, or ramucirumab. In some embodiments, the angiogenesis inhibitor is bevacizumab.

### Combinations

The phrase, “in combination with,” and the terms “co-administration,” “co-administering,” or “co-providing”, as used herein in the context of the administration of a compound described herein (e.g., a compound of Formula (I) or Formula (II)) and another agent or therapy, means that two (or more) different compounds, agents, or therapies are delivered to the subject during the course of the subject's affliction with the non-viral liver cancer, e.g., two (or more) different compounds, agents, or therapies are delivered to the subject after the subject has been diagnosed with the non-viral liver cancer and before the non-viral liver cancer has been cured or eliminated or treatment has ceased for other reasons.

In some embodiments, the delivery of one compound, agent, or therapy is still occurring when the delivery of the second begins, so that there is overlap in terms of administration. This is sometimes referred to herein as “simultaneous” or “concurrent delivery.” In other embodiments, the delivery of one compound, agent, or therapy ends before the delivery of the other compound, agent, or therapy begins. In some embodiments of either case, the treatment (e.g., administration of compound, composition, or therapy) is more effective because of combined administration. For example, the second compound, agent, or therapy is more effective, e.g., an equivalent effect is seen with less of the second compound, agent, or therapy, or the second compound, agent, or therapy reduces symptoms to a greater extent, than would be seen if the second compound, agent, or therapy were administered in the absence of the first compound, agent, or therapy, or the analogous situation is seen with the first compound, agent, or therapy. In some embodiments, delivery is such that the reduction in a symptom, or other parameter related to the disorder is greater than what would be observed with one compound, agent, or therapy delivered in the absence of the other. The effect of the two compounds or therapies can be partially additive, wholly additive, or greater than additive (e.g., synergistic). The delivery can be such that the first compound, agent, or therapy delivered is still detectable when the second is delivered.

In some embodiments, the first compound, agent, or therapy and second compound, agent, or therapy can be administered simultaneously (e.g., at the same time), in the same or in separate compositions, or sequentially. Sequential administration refers to administration of one compound, agent, or therapy before (e.g., immediately before, less than 5, 10, 15, 30, 45, 60 minutes; 1, 2, 3, 4, 6, 8, 10, 12, 16, 20, 24, 48, 72, 96 or more hours; 4, 5, 6, 7, 8, 9 or more days; 1, 2, 3, 4, 5, 6, 7, 8 or more weeks before) administration of an additional, e.g., secondary, compound, agent, or therapy. The order of administration of the first and secondary compound, agent, or therapy can also be reversed.

### Administration and Dosages

The compounds of Formula (I) or Formula (II) is administered orally, parenterally, topically, rectally, or via an implanted reservoir, preferably by oral administration. In some cases, the pH of a composition (e.g., pharmaceutical composition) comprising a compound of Formula (I) or Formula (II) is adjusted with pharmaceutically acceptable acids, bases or buffers to enhance the stability or efficacy of the composition.

In some embodiments, the subject is administered a composition (e.g., pharmaceutical composition) comprising a compound of Formula (I) or Formula (II) orally. In some embodiments the composition (e.g., pharmaceutical composition) is orally administered in any orally acceptable dosage form including, but not limited to, liquid-gel tablets or capsules, syrups, emulsions and aqueous suspensions. Liqui-gels, in some embodiments, include gelatins, plasticisers, and/or opacifiers, as needed to achieve a suitable consistency and in some embodiments, are coated with enteric coatings that are approved for use, e.g., shellacs. Additional thickening agents, for example gums, e.g., xanthum gum, starches, e.g., corn starch, or glutens in some embodiments be added to achieve a desired consistency of the composition (e.g., pharmaceutical composition) when used as an oral dosage. If desired, certain sweetening and/or flavoring and/or coloring agents, in some embodiments, are added.

In some embodiments, the subject is administered a composition (e.g., pharmaceutical composition) comprising compound of Formula (I) or Formula (II) in a form suitable for oral administration such as a tablet, capsule, pill, powder, sustained release formulations, solution, and suspension. The composition (e.g., pharmaceutical composition), in some embodiments, is in unit dosage forms suitable for single administration of precise dosages. Pharmaceutical compositions, in some embodiments, comprise, in addition to a compound as described herein a pharmaceutically acceptable carrier, and optionally further comprise one or more pharmaceutically acceptable excipients, such as, for example, stabilizers, diluents, binders, and lubricants. In addition, the tablet, in some embodiments, include other medicinal or pharmaceutical agents, carriers, and or adjuvants.

The dosage vary depending upon the dosage form employed and the route of administration utilized. The exact formulation, route of administration and dosage can be chosen by the individual physician in view of the patient's condition. Lower or higher doses than those recited above may be required. Specific dosage and treatment regimens for any particular subject will depend upon a variety of factors, including the activity of the specific compound employed, the age, body weight, general health status, sex, diet, time of administration, rate of excretion, drug combination, the severity and course of the disease, condition or symptoms, the subject's disposition to the disease, condition or symptoms, and the judgment of the treating physician. A course of therapy can comprise one or more separate administrations of a compound as described herein.

In some embodiments, the compound of Formula (I) is N-(1′,2-dihydroxy-1,2′-binaphthalen-4′-yl)-4-methoxybenzenesulfonamide, and the N-(1′,2-dihydroxy-1,2′-binaphthalen-4′-yl)-4-methoxybenzenesulfonamide is administered orally to the subject as a composition comprising:
a. a therapeutically effective amount of N-(1′,2-dihydroxy-1,2′-binaphthalen-4′-yl)-4-methoxybenzenesulfonamide or a pharmaceutically acceptable salt thereof;
b. an emulsifier (e.g., glyceride);
c. a solubilizer;
d. a polyethylene glycol (PEG); and
e. a surfactant; and optionally,
f. an antioxidant.

In some embodiments, the composition comprises:
a. a therapeutically effective amount of N-(1′,2-dihydroxy-1,2′-binaphthalen-4′-yl)-4-methoxybenzenesulfonamide or a pharmaceutically acceptable salt thereof;
b. an emulsifier (e.g., glyceride), the emulsifier being present in the composition in a weight ratio of N-(1′,2-dihydroxy-1,2′-binaphthalen-4′-yl)-4-methoxybenzenesulfonamide to the emulsifier of about 1:1 to about 1:2 (e.g., about 1:1.5);
c. a solubilizer, the solubilizer being present in the composition in a weight ratio of N-(1′,2-dihvdroxy-1,2′-binaphthalen-4′-yl)-4-methoxybenzenesulfonamide to solubilizer of about 1:1 to about 1:5 (e.g., about 1:2 to about 1:4, e.g., about 1:3);
d. a polyethylene glycol (PEG), the polyethylene glycol being present in the composition in a weight ratio of N-(1′,2-dihydroxy-1,2′-binaphthalen-4′-yl)-4-methoxybenzenesulfonamide to polyethylene glycol of about 1:2 to about 1:6 (e.g., about 1:3 to about 1:5, e.g., about 1:4);
e. a surfactant, the surfactant being present in the composition in a weight ratio of N-(1′,2-dihydroxy-1,2′-binaphthalen-4′-yl)-4-methoxybenzenesulfonamide to surfactant of about 2:1 to about 1:2 (e.g., about 1:1 to about 1:2, e.g., about 1:1, e.g., about 1:2, e.g. about 1:1.2); and
f. an antioxidant, the antioxidant being present in the composition in a weight ratio of N-(1′,2-dihydroxy-1,2′-binaphthalen-4′-yl)-4-methoxybenzenesulfonamide to antioxidant of about 10:1 to about 30:1 (e.g., about 15:1 to about 25:1, e.g., about 20:1).

In some embodiments, the emulsifier is present in the composition in a weight ratio of N-(1′,2-dihydroxy-1,2′-binaphthalen-4′-yl)-4-methoxybenzenesulfonamide to the emulsifier of about 1:1. In some embodiments, the emulsifier is present in the composition in a weight ratio of N-(1′,2-dihydroxy-1,2′-binaphthalen-4′-yl)-4-methoxybenzenesulfonamide to the emulsifier of about 1:2. In some embodiments, the emulsifier is present in the composition in a weight ratio of N-(1′,2-dihydroxy-1,2′-binaphthalen-4′-yl)-4-methoxybenzenesulfonamide to the emulsifier of about 1:3. In some embodiments, the emulsifier is present in the composition in a weight ratio of N-(1′,2-dihydroxy-1,2′-binaphthalen-4′-yl)-4-methoxybenzenesulfonamide to the emulsifier of about 1:4. In some embodiments, the emulsifier is present in the composition in a weight ratio of N-(1′,2-dihydroxy-1,2′-binaphthalen-4′-yl)-4-methoxybenzenesulfonamide to the emulsifier of about 1:5. In some embodiments, the emulsifier is present in the composition in a weight ratio of N-(1′,2-dihydroxy-1,2′-binaphthalen-4′-yl)-4-methoxybenzenesulfonamide to the emulsifier of about 1:6. In some embodiments, the emulsifier is present in the composition in a weight ratio of N-(1′,2-dihydroxy-1,2′-binaphthalen-4′-yl)-4-methoxybenzenesulfonamide to the emulsifier of about 1:7. In some embodiments, the emulsifier is present in the composition in a weight ratio of N-(1′,2-dihydroxy-1,2′-binaphthalen-4′-yl)-4-methoxybenzenesulfonamide to the emulsifier of about 1:1.5.

In some embodiments, the solubilizer is present in the composition in a weight ratio of N-(1′,2-dihydroxy-1,2′-binaphthalen-4′-yl)-4-methoxybenzenesulfonamide to solubilizer of about 1:1. In some embodiments, the solubilizer is present in the composition in a weight ratio of N-(1′,2-dihydroxy-1,2′-binaphthalen-4′-yl)-4-methoxybenzenesulfonamide to solubilizer of about 1:2. In some embodiments, the solubilizer is present in the composition in a weight ratio of N-(1′,2-dihydroxy-1,2′-binaphthalen-4′-yl)-4-methoxybenzenesulfonamide to solubilizer of about 1:3. In some embodiments, the solubilizer is present in the composition in a weight ratio of N-(1′,2-dihydroxy-1,2′-binaphthalen-4′-yl)-4-methoxybenzenesulfonamide to solubilizer of about 1:4. In some embodiments, the solubilizer is present in the composition in a weight ratio of N-(1′,2-dihydroxy-1,2′-binaphthalen-4′-yl)-4-methoxybenzenesulfonamide to solubilizer of about 1:5. In some embodiments, the solubilizer is present in the composition in a weight ratio of N-(1′,2-dihydroxy-1,2′-binaphthalen-4′-yl)-4-methoxybenzenesulfonamide to solubilizer of about 1:4. In some embodiments, the solubilizer is present in the composition in a weight ratio of N-(1′,2-dihydroxy-1,2′-binaphthalen-4′-yl)-4-methoxybenzenesulfonamide to solubilizer of about 1:6. In some embodiments, the solubilizer is present in the composition in a weight ratio of N-(1′,2-dihydroxy-1,2′-binaphthalen-4′-yl)-4-methoxybenzenesulfonamide to solubilizer of about 1:4. In some embodiments, the solubilizer is present in the composition in a weight ratio of N-(1′,2-dihydroxy-1,2′-binaphthalen-4′-yl)-4-methoxybenzenesulfonamide to solubilizer of about 1:7. In some embodiments, the solubilizer is present in the composition in a weight ratio of N-(1′,2-dihydroxy-1,2′-binaphthalen-4′-yl)-4-methoxybenzenesulfonamide to solubilizer of about 1:4. In some embodiments, the solubilizer is present in the composition in a weight ratio of N-(1′,2-dihydroxy-1,2′-binaphthalen-4′-yl)-4-methoxybenzenesulfonamide to solubilizer of about 1:8. In some embodiments, the solubilizer is present in the composition in a weight ratio of N-(1′,2-dihydroxy-1,2′-binaphthalen-4′-yl)-4-methoxybenzenesulfonamide to solubilizer of about 1:4. In some embodiments, the solubilizer is present in the composition in a weight ratio of N-(1′,2-dihydroxy-1,2′-binaphthalen-4′-yl)-4-methoxybenzenesulfonamide to solubilizer of about 1:9. In some embodiments, the solubilizer is present in the composition in a weight ratio of N-(1′,2-dihydroxy-1,2′-binaphthalen-4′-yl)-4-methoxybenzenesulfonamide to solubilizer of about 1:10. In some embodiments, the solubilizer is present in the composition in a weight ratio of N-(1′,2-dihydroxy-1,2′-binaphthalen-4′-yl)-4-methoxybenzenesulfonamide to solubilizer of about 1:11. In some embodiments, the solubilizer is present in the composition in a weight ratio of N-(1′,2-dihydroxy-1,2′-binaphthalen-4′-yl)-4-methoxybenzenesulfonamide to solubilizer of about 1:12. In some embodiments, the solubilizer is present in the composition in a weight ratio of N-(1′,2-dihydroxy-1,2′-binaphthalen-4′-yl)-4-methoxybenzenesulfonamide to solubilizer of about 1:13. In some embodiments, the solubilizer is present in the composition in a weight ratio of N-(1′,2-dihydroxy-1,2′-binaphthalen-4′-yl)-4-methoxybenzenesulfonamide to solubilizer of about 1:14. In some embodiments, the solubilizer is present in the composition in a weight ratio of N-(1′,2-dihydroxy-1,2′-binaphthalen-4′-yl)-4-methoxybenzenesulfonamide to solubilizer of about 1:15. In some embodiments, the solubilizer is present in the composition in a weight ratio of N-(1′,2-dihydroxy-1,2′-binaphthalen-4′-yl)-4-methoxybenzenesulfonamide to solubilizer of about 1:16. In some embodiments, the solubilizer is present in the composition in a weight ratio of N-(1′,2-dihydroxy-1,2′-binaphthalen-4′-yl)-4-methoxybenzenesulfonamide to solubilizer of about 1:17. In some embodiments, the solubilizer is present in the composition in a weight ratio of N-(1′,2-dihydroxy-1,2′-binaphthalen-4′-yl)-4-methoxybenzenesulfonamide to solubilizer of about 1:18. In some embodiments, the solubilizer is present in the composition in a weight ratio of N-(1′,2-dihydroxy-1,2′-binaphthalen-4′-yl)-4-methoxybenzenesulfonamide to solubilizer of about 1:19. In some embodiments, the solubilizer is present in the composition in a weight ratio of N-(1′,2-dihydroxy-1,2′-binaphthalen-4′-yl)-4-methoxybenzenesulfonamide to solubilizer of about 1:20.

In some embodiments, the polyethylene glycol is present in the composition in a weight ratio of N-(1′,2-dihydroxy-1,2′-binaphthalen-4′-yl)-4-methoxybenzenesulfonamide to polyethylene glycol of about 1:1. In some embodiments, the polyethylene glycol is present in the composition in a weight ratio of N-(1′,2-dihydroxy-1,2′-binaphthalen-4′-yl)-4-methoxybenzenesulfonamide to polyethylene glycol of about 1:2. In some embodiments, the polyethylene glycol is present in the composition in a weight ratio of N-(1′,2-dihydroxy-1,2′-binaphthalen-4′-yl)-4-methoxybenzenesulfonamide to polyethylene glycol of about 1:3. In some embodiments, the polyethylene glycol is present in the composition in a weight ratio of N-(1′,2-dihydroxy-1,2′-binaphthalen-4′-yl)-4-methoxybenzenesulfonamide to polyethylene glycol of about 1:4. In some embodiments, the polyethylene glycol is present in the composition in a weight ratio of N-(1′,2-dihydroxy-1,2′-binaphthalen-4′-yl)-4-methoxybenzenesulfonamide to polyethylene glycol of about 1:4.2. In some embodiments, the polyethylene glycol is present in the composition in a weight ratio of N-(1′,2-dihydroxy-1,2′-binaphthalen-4′-yl)-4-methoxybenzenesulfonamide to polyethylene glycol of about 1:5. In some embodiments, the polyethylene glycol is present in the composition in a weight ratio of N-(1′,2-dihydroxy-1,2′-binaphthalen-4′-yl)-4-methoxybenzenesulfonamide to polyethylene glycol of about 1:6. In some embodiments, the polyethylene glycol is present in the composition in a weight ratio of N-(1′,2-dihydroxy-1,2′-binaphthalen-4′-yl)-4-methoxybenzenesulfonamide to polyethylene glycol of about 1:7. In some embodiments, the polyethylene glycol is present in the composition in a weight ratio of N-(1′,2-dihydroxy-1,2′-binaphthalen-4′-yl)-4-methoxybenzenesulfonamide to polyethylene glycol of about 1:8. In some embodiments, the polyethylene glycol is present in the composition in a weight ratio of N-(1′,2-dihydroxy-1,2′-binaphthalen-4′-yl)-4-methoxybenzenesulfonamide to polyethylene glycol of about 1:9. In some embodiments, the polyethylene glycol is present in the composition in a weight ratio of N-(1′,2-dihydroxy-1,2′-binaphthalen-4′-yl)-4-methoxybenzenesulfonamide to polyethylene glycol of about 1:10. In some embodiments, the polyethylene glycol is present in the composition in a weight ratio of N-(1′,2-dihydroxy-1,2′-binaphthalen-4′-yl)-4-methoxybenzenesulfonamide to polyethylene glycol of about 1:11. In some embodiments, the polyethylene glycol is present in the composition in a weight ratio of N-(1′,2-dihydroxy-1,2′-binaphthalen-4′-yl)-4-methoxybenzenesulfonamide to polyethylene glycol of about 1:12. In some embodiments, the polyethylene glycol is present in the composition in a weight ratio of N-(1′,2-dihydroxy-1,2′-binaphthalen-4′-yl)-4-methoxybenzenesulfonamide to polyethylene glycol of about 1:13. In some embodiments, the polyethylene glycol is present in the composition in a weight ratio of N-(1′,2-dihvdroxy-1,2′-binaphthalen-4′-yl)-4-methoxybenzenesulfonamide to polyethylene glycol of about 1:14. In some embodiments, the polyethylene glycol is present in the composition in a weight ratio of N-(1′,2-dihydroxy-1,2′-binaphthalen-4′-yl)-4-methoxybenzenesulfonamide to polyethylene glycol of about 1:15. In some embodiments, the polyethylene glycol is present in the composition in a weight ratio of N-(1′,2-dihydroxy-1,2′-binaphthalen-4′-yl)-4-methoxybenzenesulfonamide to polyethylene glycol of about 1:16. In some embodiments, the polyethylene glycol is present in the composition in a weight ratio of N-(1′,2-dihydroxy-1,2′-binaphthalen-4′-yl)-4-methoxybenzenesulfonamide to polyethylene glycol of about 1:17. In some embodiments, the polyethylene glycol is present in the composition in a weight ratio of N-(1′,2-dihydroxy-1,2′-binaphthalen-4′-yl)-4-methoxybenzenesulfonamide to polyethylene glycol of about 1:18. In some embodiments, the polyethylene glycol is present in the composition in a weight ratio of N-(1′,2-dihydroxy-1,2′-binaphthalen-4′-yl)-4-methoxybenzenesulfonamide to polyethylene glycol of about 1:19. In some embodiments, the polyethylene glycol is present in the composition in a weight ratio of N-(1′,2-dihydroxy-1,2′-binaphthalen-4′-yl)-4-methoxybenzenesulfonamide to polyethylene glycol of about 1:20.

In some embodiments, the surfactant is present in the composition in a weight ratio of N-(1′,2-dihydroxy-1,2′-binaphthalen-4′-yl)-4-methoxybenzenesulfonamide to surfactant of about 4:1. In some embodiments, the surfactant is present in the composition in a weight ratio of N-(1′,2-dihydroxy-1,2′-binaphthalen-4′-yl)-4-methoxybenzenesulfonamide to surfactant of about 3:1. In some embodiments, the surfactant is present in the composition in a weight ratio of N-(1′,2-dihydroxy-1,2′-binaphthalen-4′-yl)-4-methoxybenzenesulfonamide to surfactant of about 2:1. In some embodiments, the surfactant is present in the composition in a weight ratio of N-(1′,2-dihydroxy-1,2′-binaphthalen-4′-yl)-4-methoxybenzenesulfonamide to surfactant of about 1:1. In some embodiments, the surfactant is present in the composition in a weight ratio of N-(1′,2-dihydroxy-1,2′-binaphthalen-4′-yl)-4-methoxybenzenesulfonamide to surfactant of about 1:1.2. In some embodiments, the surfactant is present in the composition in a weight ratio of N-(1′,2-dihydroxy-1,2′-binaphthalen-4′-yl)-4-methoxybenzenesulfonamide to surfactant of about 1:2. In some embodiments, the surfactant is present in the composition in a weight ratio of N-(1′,2-dihydroxy-1,2′-binaphthalen-4′-yl)-4-methoxybenzenesulfonamide to surfactant of about 1:3. In some embodiments, the surfactant is present in the composition in a weight ratio of N-(1′,2-dihydroxy-1,2′-binaphthalen-4′-yl)-4-methoxybenzenesulfonamide to surfactant of about 1:4. In some embodiments, the surfactant is present in the composition in a weight ratio of N-(1′,2-dihydroxy-1,2′-binaphthalen-4′-yl)-4-methoxybenzenesulfonamide to surfactant of about 1:5. In some embodiments, the surfactant is present in the composition in a weight ratio of N-(1′,2-dihydroxy-1,2′-binaphthalen-4′-yl)-4-methoxybenzenesulfonamide to surfactant of about 1:6.

In some embodiments, the antioxidant is present in the composition in a weight ratio of N-(1′,2-dihydroxy-1,2′-binaphthalen-4′-yl)-4-methoxybenzenesulfonamide to antioxidant of about 2:1. In some embodiments, the antioxidant is present in the composition in a weight ratio of N-(1′,2-dihydroxy-1,2′-binaphthalen-4′-yl)-4-methoxybenzenesulfonamide to antioxidant of about 3:1. In some embodiments, the antioxidant is present in the composition in a weight ratio of N-(1′,2-dihydroxy-1,2′-binaphthalen-4′-yl)-4-methoxybenzenesulfonamide to antioxidant of about 4:1. In some embodiments, the antioxidant is present in the composition in a weight ratio of N-(1′,2-dihydroxy-1,2′-binaphthalen-4′-yl)-4-methoxy benzenesulfonamide to antioxidant of about 5:1. In some embodiments, the antioxidant is present in the composition in a weight ratio of N-(1′,2-dihydroxy-1,2′-binaphthalen-4′-yl)-4-methoxybenzenesulfonamide to antioxidant of about 6:1. In some embodiments, the antioxidant is present in the composition in a weight ratio of N-(1′,2-dihydroxy-1,2′-binaphthalen-4′-yl)-4-methoxybenzenesulfonamide to antioxidant of about 7:1. In some embodiments, the antioxidant is present in the composition in a weight ratio of N-(1′,2-dihydroxy-1,2′-binaphthalen-4′-yl)-4-methoxybenzenesulfonamide to antioxidant of about 8:1. In some embodiments, the antioxidant is present in the composition in a weight ratio of N-(1′,2-dihydroxy-1,2′-binaphthalen-4′-yl)-4-methoxybenzenesulfonamide to antioxidant of about 9:1. In some embodiments, the antioxidant is present in the composition in a weight ratio of N-(1′,2-dihydroxy-1,2′-binaphthalen-4′-yl)-4-methoxybenzenesulfonamide to antioxidant of about 10:1. In some embodiments, the antioxidant is present in the composition in a weight ratio of N-(1′,2-dihydroxy-1,2′-binaphthalen-4′-yl)-4-methoxybenzenesulfonamide to antioxidant of about 11:1. In some embodiments, the antioxidant is present in the composition in a weight ratio of N-(1′,2-dihydroxy-1,2′-binaphthalen-4′-yl)-4-methoxybenzenesulfonamide to antioxidant of about 12:1. In some embodiments, the antioxidant is present in the composition in a weight ratio of N-(1′,2-dihydroxy-1,2′-binaphthalen-4′-yl)-4-methoxybenzenesulfonamide to antioxidant of about 13:1. In some embodiments, the antioxidant is present in the composition in a weight ratio of N-(1′,2-dihydroxy-1,2′-binaphthalen-4′-yl)-4-methoxybenzenesulfonamide to antioxidant of about 14:1. In some embodiments, the antioxidant is present in the composition in a weight ratio of N-(1′,2-dihydroxy-1,2′-binaphthalen-4′-yl)-4-methoxybenzenesulfonamide to antioxidant of about 15:1. In some embodiments, the antioxidant is present in the composition in a weight ratio of N-(1′,2-dihydroxy-1,2′-binaphthalen-4′-yl)-4-methoxybenzenesulfonamide to antioxidant of about 16:1. In some embodiments, the antioxidant is present in the composition in a weight ratio of N-(1′,2-dihydroxy-1,2′-binaphthalen-4′-yl)-4-methoxybenzenesulfonamide to antioxidant of about 17:1. In some embodiments, the antioxidant is present in the composition in a weight ratio of N-(1′,2-dihydroxy-1,2′-binaphthalen-4′-yl)-4-methoxybenzenesulfonamide to antioxidant of about 18:1. In some embodiments, the antioxidant is present in the composition in a weight ratio of N-(1′,2-dihydroxy-1,2′-binaphthalen-4′-yl)-4-methoxybenzenesulfonamide to antioxidant of about 18.4:1. In some embodiments, the antioxidant is present in the composition in a weight ratio of N-(1′,2-dihydroxy-1,2′-binaphthalen-4′-yl)-4-methoxybenzenesulfonamide to antioxidant of about 19:1. In some embodiments, the antioxidant is present in the composition in a weight ratio of N-(1′,2-dihydroxy-1,2′-binaphthalen-4′-yl)-4-methoxybenzenesulfonamide to antioxidant of about 20:1. In some embodiments, the antioxidant is present in the composition in a weight ratio of N-(1′,2-dihydroxy-1,2′-binaphthalen-4′-yl)-4-methoxybenzenesulfonamide to antioxidant of about 21:1. In some embodiments, the antioxidant is present in the composition in a weight ratio of N-(1′,2-dihydroxy-1,2′-binaphthalen-4′-yl)-4-methoxybenzenesulfonamide to antioxidant of about 22:1. In some embodiments, the antioxidant is present in the composition in a weight ratio of N-(1′,2-dihydroxy-1,2′-binaphthalen-4′-yl)-4-methoxybenzenesulfonamide to antioxidant of about 23:1. In some embodiments, the antioxidant is present in the composition in a weight ratio of N-(1′,2-dihydroxy-1,2′-binaphthalen-4′-yl)-4-methoxybenzenesulfonamide to antioxidant of about 24:1. In some embodiments, the antioxidant is present in the composition in a weight ratio of N-(1′,2-dihydroxy-1,2′-binaphthalen-4′-yl)-4-methoxybenzenesulfonamide to antioxidant of about 25:1. In some embodiments, the antioxidant is present in the composition in a weight ratio of N-(1′,2-dihydroxy-1,2′-binaphthalen-4′-yl)-4-methoxybenzenesulfonamide to antioxidant of about 26:1. In some embodiments, the antioxidant is present in the composition in a weight ratio of N-(1′,2-dihydroxy-1,2′-binaphthalen-4′-yl)-4-methoxybenzenesulfonamide to antioxidant of about 27:1. In some embodiments, the antioxidant is present in the composition in a weight ratio of N-(1′,2-dihydroxy-1,2′-binaphthalen-4′-yl)-4-methoxybenzenesulfonamide to antioxidant of about 28:1. In some embodiments, the antioxidant is present in the composition in a weight ratio of N-(1′,2-dihydroxy-1,2′-binaphthalen-4′-yl)-4-methoxy benzenesulfonamide to antioxidant of about 29:1. In some embodiments, the antioxidant is present in the composition in a weight ratio of N-(1′,2-dihydroxy-1,2′-binaphthalen-4′-yl)-4-methoxybenzenesulfonamide to antioxidant of about 30:1.

In some embodiments, the N-(1′,2-dihydroxy-1,2′-binaphthalen-4′-yl)-4-methoxybenzenesulfonamide is present in the composition in a concentration of at least 50 mg/g (e.g., at least 60 mg/g, at least 70 mg/g, at least 80 mg/g, or at least 90 mg/g) (e.g., excluding the mass of a capsule shell). In some embodiments, the N-(1′,2-dihydroxy-1,2′-binaphthalen-4′-yl)-4-methoxybenzenesulfonamide is present in the composition in a concentration of at least 50 mg/g. In some embodiments, the N-(1′,2-dihydroxy-1,2′-binaphthalen-4′-yl)-4-methoxybenzenesulfonamide is present in the composition in a concentration of at least 60 mg/g. In some embodiments, the N-(1′,2-dihydroxy-1,2′-binaphthalen-4′-yl)-4-methoxybenzenesulfonamide is present in the composition in a concentration of at least 70 mg/g. In some embodiments, the N-(1′,2-dihydroxy-1,2′-binaphthalen-4′-yl)-4-methoxybenzenesulfonamide is present in the composition in a concentration of at least 80 mg/g. In some embodiments, the N-(1′,2-dihydroxy-1,2′-binaphthalen-4′-yl)-4-methoxybenzenesulfonamide is present in the composition in a concentration of at least 90 mg/g.

In some embodiments, the emulsifier is a glyceride emulsifier. In some embodiments, the emulsifier comprises optionally polyglycolyzed medium- and/or long-chain mono-, di-, and/or tri-glyceride(s). In some embodiments, the emulsifier comprises a medium-chain mono-glyceride. In some embodiments, the emulsifier comprises a polyglycolyzed medium-chain mono-glyceride. In some embodiments, the emulsifier comprises a long-chain mono-glyceride. In some embodiments, the emulsifier comprises a polyglycolyzed long-chain mono-glyceride. In some embodiments, the emulsifier comprises a medium-chain di-glyceride. In some embodiments, the emulsifier comprises a polyglycolyzed medium-chain di-glyceride. In some embodiments, the emulsifier comprises a long-chain di-glyceride. In some embodiments, the emulsifier comprises a polyglycolyzed long-chain di-glyceride. In some embodiments, the emulsifier comprises a medium-chain tri-glyceride. In some embodiments, the emulsifier comprises a polyglycolyzed medium-chain tri-glyceride. In some embodiments, the emulsifier comprises a long-chain tri-glyceride. In some embodiments, the emulsifier comprises a polyglycolyzed long-chain tri-glyceride.

In some embodiments, the emulsifier is Labrasol®. In some embodiments, the emulsifier is CapmulC MCM. In some embodiments, the emulsifier is Capmul® MCM EP. In some embodiments, the emulsifier is Capmul® C8 EP. In some embodiments, the emulsifier is Capryol® 90.

In some embodiments, the solubilizer is a polyoxyl castor oil or a vitamin E polyethylene glycol succinate (TPGS). In some embodiments, the solubilizer is a polyoxyl castor oil. In some embodiments, the solubilizer is a vitamin E polyethylene glycol succinate. In some embodiments, the surfactant is Kolliphor® RH 40. In some embodiments, the solubilizer is Vitamin E TPGS.

In some embodiments, the polyethylene glycol (PEG) has an average molecular weight of about 200 to about 1000 (e.g., about 500 to about 700, or about 550 to about 650, or about 600). In some embodiments, the polyethylene glycol (PEG) has an average molecular weight of about 200 to 1000. In some embodiments, the polyethylene glycol (PEG) has an average molecular weight of about 500 to 700. In some embodiments, the polyethylene glycol (PEG) has an average molecular weight of about 550 to 650. In some embodiments, the polyethylene glycol (PEG) has an average molecular weight of about 600.

In some embodiments, the polyethylene glycol (PEG) is PEG200. In some embodiments, the polyethylene glycol (PEG) is PEG300. In some embodiments, the polyethylene glycol (PEG) is PEG400. In some embodiments, the polyethylene glycol (PEG) is PEG500. In some embodiments, the polyethylene glycol (PEG) is PEG600. In some embodiments, the polyethylene glycol (PEG) is PEG700. In some embodiments, the polyethylene glycol (PEG) is PEG800. In some embodiments, the polyethylene glycol (PEG) is PEG900. In some embodiments, the polyethylene glycol (PEG) is PEG1000.

In some embodiments, the surfactant is polysorbate (e.g., polysorbate 20). In some embodiments, the surfactant is polysorbate 20. In some embodiments, the surfactant is polysorbate 40. In some embodiments, the surfactant is polysorbate 60. In some embodiments, the surfactant is polysorbate 80.

In some embodiments, the antioxidant is vitamin E. In some embodiments, the antioxidant is ascorbyl palmitate. In some embodiments, the antioxidant is butylated hydroxytoluene. In some embodiments, the antioxidant is triethyl citrate. In some embodiments, the antioxidant is citric acid.

In some embodiments, the composition further comprises a co-solvent (e.g., Transcutol®). In some embodiments, the composition further comprises Transcutol® HP.

In some embodiments, the methods described herein contemplate administering to the subject an oral dosage form comprising the composition as described herein contained within a capsule.

In some embodiments, the compound of Formula (I) is N-(1′,2-dihydroxy-1,2′-binaphthalen-4′-yl)-4-methoxybenzenesulfonamide, and the N-(1′,2-dihydroxy-1,2′-binaphthalen-4′-yl)-4-methoxybenzenesulfonamide, is orally administered to the subject as a composition comprising: a solid amorphous dispersion comprising: a) a therapeutically effective amount of N-(1′,2-dihydroxy-1,2′-binaphthalen-4′-yl)-4-methoxybenzenesulfonamide, or a pharmaceutically acceptable salt thereof; and b) a polymer excipient.

In some embodiments, the composition further comprises a crystallization inhibiting polymer. In some embodiments, the composition further comprises at least one of: diluent or filler, disintegrant, or lubricant.

In some embodiments, the composition comprises:
a) a solid amorphous dispersion comprising:
   i) an effective amount of N-(1′,2-dihydroxy-1,2′-binaphthalen-4′-yl)-4-methoxybenzenesulfonamide, or a pharmaceutically acceptable salt thereof (e.g., at least 150 mg of N-(1′,2-dihydroxy-1,2′-binaphthalen-4′-yl)-4-methoxybenzenesulfonamide, or a pharmaceutically acceptable salt thereof); and
   ii) a polymer excipient in an amount of about 15-25% w/w of the composition;
b) a crystallization inhibiting polymer in an amount of about 15-25% w/w of the composition;
c) a diluent or filler in an amount of about 20-35% of the composition;
d) a disintegrant in an amount of about 3-8% w/w of the composition; and
e) a lubricant in an amount of about 1-3% w/w of the composition.

In some embodiments, the polymer excipient is one or more celluloses or derivatives thereof (e.g., microcrystalline cellulose, carboxymethylcellulose, hydroxypropyl methylcellulose (HPMC) or hvpromellose, hydroxypropyl methylcellulose acetate succinate (HPMCAS) or hypromellose acetate succinate (e.g., HPMCAS-LG, HPMCAS-MG, HPMCAS-HG), HPMC phthalate (HPMCP) (e.g., HPMCP-HP55, HPMCP-HP55S), Methocel™ (e.g., Methocel™ E3 (e.g., Methocel™ E3LV))), hydroxypropyl cellulose, hydroxyethylcellulose, ethylcellulose, and cellulose acetate phthalate), polyacrylates (e.g., polymethacrylates (e.g., copolymers comprising methacrylic acid and methyl methacrylate, copolymers comprising methacrylic acid and ethylacrylate, copolymer comprising N,N-dimethylaminoethyl methacrylate, methylmethacrylate, and butylmethacrylate (e.g., Eudragit®, (e.g., Eduragit® EPO, Eudragit® L30 D-55, Eudragit® L100, Eudragit® L100-55)))), polyvinyl pyrrolidones (PVP), polyvinyl pyrrolidone vinyl acetates (PVPVA), other copolymers (e.g., copolymers comprising polyethylene glycol, polyvinylcaprolactam, and polyvinylacetate (e.g., polyvinyl acetate phthalate (PVAP); polyvinyl caprolactam-polyvinyl acetate-polyethylene glycol graft copolymer)) (e.g., Soluplus®), or copolymers comprising vinyl acetate and N-vinyl-2-pyrrolidone (e.g., Plasdone™ (e.g., Plasdone™ 5630 (e.g., Plasdone™ S630 Ultra)), or mixtures thereof.

In some embodiments, the polymer excipient is selected from: hydroxypropyl methylcellulose (HPMC), a copolymer comprising methacrylic acid, a copolymer comprising vinyl acetate, and N-vinyl-2-pyrrolidone, and combinations thereof.

In some embodiments, the polymer excipient is hydroxypropyl methylcellulose acetate succinate (HPMC AS). In some embodiments, the HPMC AS is HPMC AS L grade (HPMC AS-L).

In some embodiments, the polymer excipient is a copolymer comprising methacrylic acid and ethylacrylate, a copolymer comprising methacrylic acid and methyl methacrylate, or a copolymer comprising N,N-dimethylaminoethyl methacrylate, methylmethacrylate, and butylmethacrylate. In some embodiments, the polymer excipient is a copolymer comprising methacrylic acid and ethylacrylate. In some embodiments, the polymer excipient is a copolymer of about 1.4:1 to about 1:1.4 methacrylic acid and ethylacrylate. In some embodiments, the polymer excipient is a copolymer of about 1.2:1 to about 1:1.2 methacrylic acid and ethylacrylate. In some embodiments, the polymer excipient is a copolymer of 1:1 methacrylic acid and ethylacrylate.

The polymer excipient, in some embodiments, also act as a crystallization inhibitor, which helps slow or inhibit crystallization of TTI-101 when it is released from the formulation in the GI tract or mediates supersaturation stabilization (e.g., stabilization of a supersaturated solution).

In some embodiments, the solid amorphous dispersion comprises about 40% w/w to about 80% w/w of the N-(1′,2-dihydroxy-1,2′-binaphthalen-4′-yl)-4-methoxybenzenesulfonamide. In some embodiments, the solid amorphous dispersion comprises about 40% w/w to about 70% w/w of the N-(1′,2-dihydroxy-1,2′-binaphthalen-4′-yl)-4-methoxybenzenesulfonamide. In some embodiments, the solid amorphous dispersion comprises about 40% w/w to about 60% w/w of the N-(1′,2-dihydroxy-1,2′-binaphthalen-4′-yl)-4-methoxybenzenesulfonamide. In some embodiments, the solid amorphous dispersion comprises about 45% w/w to about 55% w/w of the N-(1′,2-dihydroxy-1,2′-binaphthalen-4′-yl)-4-methoxybenzenesulfonamide. In some embodiments, the solid amorphous dispersion comprises about 40% w/w of the N-(1′,2-dihydroxy-1,2′-binaphthalen-4′-yl)-4-methoxybenzenesulfonamide. In some embodiments, the solid amorphous dispersion comprises about 45% w/w of the N-(1′,2-dihydroxy-1,2′-binaphthalen-4′-yl)-4-methoxybenzenesulfonamide. In some embodiments, the solid amorphous dispersion comprises about 50% w/w of the N-(1′,2-dihydroxy-1,2′-binaphthalen-4′-yl)-4-methoxybenzenesulfonamide. In some embodiments, the solid amorphous dispersion comprises about 55% w/w of the N-(1′,2-dihydroxy-1,2′-binaphthalen-4′-yl)-4-methoxybenzenesulfonamide. In some embodiments, the solid amorphous dispersion comprises about 60% w/w of the N-(1′,2-dihydroxy-1,2′-binaphthalen-4′-yl)-4-methoxybenzenesulfonamide. In some embodiments, the solid amorphous dispersion comprises about 65% w/w of the N-(1′,2-dihydroxy-1,2′-binaphthalen-4′-yl)-4-methoxybenzenesulfonamide. In some embodiments, the solid amorphous dispersion comprises about 70% w/w of the N-(1′,2-dihydroxy-1,2′-binaphthalen-4′-yl)-4-methoxybenzenesulfonamide. In some embodiments, the solid amorphous dispersion comprises about 75% w/w of the N-(1′,2-dihydroxy-1,2′-binaphthalen-4′-yl)-4-methoxybenzenesulfonamide. In some embodiments, the solid amorphous dispersion comprises about 80% w/w of the N-(1′,2-dihydroxy-1,2′-binaphthalen-4′-yl)-4-methoxybenzenesulfonamide.

In some embodiments, the solid amorphous dispersion comprises about 40% w/w to about 80% w/w of the polymer excipient. In some embodiments, the solid amorphous dispersion comprises In some embodiments, the solid amorphous dispersion comprises about 40% w/w to about 70% w/w of the polymer excipient. In some embodiments, the solid amorphous dispersion comprises about 40% w/w to about 60% w/w of the polymer excipient. In some embodiments, the solid amorphous dispersion comprises about 45% w/w to about 55% w/w of the polymer excipient. In some embodiments, the solid amorphous dispersion comprises about 40% w/w of the polymer excipient. In some embodiments, the solid amorphous dispersion comprises about 45% w/w of the polymer excipient. In some embodiments, the solid amorphous dispersion comprises about 50% w/w of the polymer excipient. In some embodiments, the solid amorphous dispersion comprises about 55% w/w of the polymer excipient. In some embodiments, the solid amorphous dispersion comprises about 60% w/w of the polymer excipient. In some embodiments, the solid amorphous dispersion comprises about 65% w/w of the polymer excipient. In some embodiments, the solid amorphous dispersion comprises about 70% w/w of the polymer excipient. In some embodiments, the solid amorphous dispersion comprises about 75% w/w of the polymer excipient. In some embodiments, the solid amorphous dispersion comprises about 80% w/w of the polymer excipient.

In some embodiments, the solid amorphous dispersion further comprises an antioxidant.

In some embodiments, the antioxidant is vitamin E. In some embodiments, the antioxidant is ascorbyl palmitate. In some embodiments, the antioxidant is butylated hydroxytoluene. In some embodiments, the antioxidant is triethyl citrate. In some embodiments, the antioxidant is citric acid. In some embodiments, the antioxidant is ascorbic acid.

In some embodiments, the antioxidant is present in the solid amorphous dispersion in a % w/w of about 0.05 to about 5% (e.g., about 0.1 to about 3%, about 0.2 to about 1%). In some embodiments, the antioxidant is present in the solid amorphous dispersion in a % w/w of about 0.05%. In some embodiments, the antioxidant is present in the solid amorphous dispersion in a % w/w of about 0.1%. In some embodiments, the antioxidant is present in the solid amorphous dispersion in a % w/w of about 0.2%. In some embodiments, the antioxidant is present in the solid amorphous dispersion in a % w/w of about 0.3%. In some embodiments, the antioxidant is present in the solid amorphous dispersion in a % w/w of about 0.4%. In some embodiments, the antioxidant is present in the solid amorphous dispersion in a % w/w of about 0.5%. In some embodiments, the antioxidant is present in the solid amorphous dispersion in a % w/w of about 0.6%. In some embodiments, the antioxidant is present in the solid amorphous dispersion in a % w/w of about 0.7%. In some embodiments, the antioxidant is present in the solid amorphous dispersion in a % w/w of about 0.8%. In some embodiments, the antioxidant is present in the solid amorphous dispersion in a % w/w of about 0.9%. In some embodiments, the antioxidant is present in the solid amorphous dispersion in a % w/w of about 1%. In some embodiments, the antioxidant is present in the solid amorphous dispersion in a % w/w of about 2%. In some embodiments, the antioxidant is present in the solid amorphous dispersion in a % w/w of about 3%. In some embodiments, the antioxidant is present in the solid amorphous dispersion in a % w/w of about 4%. In some embodiments, the antioxidant is present in the solid amorphous dispersion in a % w/w of about 5%.

In some embodiments, the solid amorphous dispersion further comprises a surfactant.

In some embodiments, the surfactant is a copolymer comprising polyoxypropylene and polyoxyethylene, polyoxyl 40 hydrogenated castor oil, or a water-soluble derivative of natural Vitamin E (e.g., D-α-tocopheryl polyethylene glycol succinate (vitamin E TPGS)). In some embodiments, the surfactant is poloxamer 188, poloxamer 407, Kolliphor® RH40, or vitamin E TPGS.

In some embodiments, the surfactant is present in the solid amorphous dispersion in a % w/w of about 0.01 to about 10%. In some embodiments, the surfactant is present in the solid amorphous dispersion in a % w/w of about 0.01 to about 5%. In some embodiments, the surfactant is present in the solid amorphous dispersion in a % w/w of about 0.1 to about 5%. In some embodiments, the surfactant is present in the solid amorphous dispersion in a % w/w of about 0.5 to about 5%. In some embodiments, the surfactant is present in the solid amorphous dispersion in a % w/w of about 1 to about 5%. In some embodiments, the surfactant is present in the solid amorphous dispersion in a % w/w of about 1%. In some embodiments, the surfactant is present in the solid amorphous dispersion in a % w/w of about 2%. In some embodiments, the surfactant is present in the solid amorphous dispersion in a % w/w of about 3%. In some embodiments, the surfactant is present in the solid amorphous dispersion in a % w/w of about 4%. In some embodiments, the surfactant is present in the solid amorphous dispersion in a % w/w of about 5%.

In some embodiments, the composition comprises about 5% w/w to about 50% w/w of N-(1′,2-dihydroxy-1,2′-binaphthalen-4′-yl)-4-methoxybenzenesulfonamide. In some embodiments, the composition comprises about 5% w/w to about 40% w/w of N-(1′,2-dihydroxy-1,2′-binaphthalen-4′-yl)-4-methoxybenzenesulfonamide. In some embodiments, the composition comprises about 10% w/w to about 40% w/w of N-(1′,2-dihydroxy-1,2′-binaphthalen-4′-yl)-4-methoxy benzenesulfonamide. In some embodiments, the composition comprises about 15% w/w to about 40% w/w of N-(1′,2-dihydroxy-1,2′-binaphthalen-4′-yl)-4-methoxybenzenesulfonamide. In some embodiments, the composition comprises about 20% w/w to about 40% w/w of N-(1′,2-dihydroxy-1,2′-binaphthalen-4′-yl)-4-methoxybenzenesulfonamide. In some embodiments, the composition comprises about 5% w/w to about 30% w/w of N-(1′,2-dihydroxy-1,2′-binaphthalen-4′-yl)-4-methoxybenzenesulfonamide. In some embodiments, the composition comprises about 10% w/w to about 30% w/w of N-(1′,2-dihydroxy-1,2′-binaphthalen-4′-yl)-4-methoxybenzenesulfonamide. In some embodiments, the composition comprises about 20% w/w to about 35% w/w of N-(1′,2-dihydroxy-1,2′-binaphthalen-4′-yl)-4-methoxybenzenesulfonamide. In some embodiments, the composition comprises about 25% w/w to about 35% w/w of N-(1′,2-dihydroxy-1,2′-binaphthalen-4′-yl)-4-methoxybenzenesulfonamide. In some embodiments, the composition comprises about 15% w/w to about 30% w/w of N-(1′,2-dihydroxy-1,2′-binaphthalen-4′-yl)-4-methoxybenzenesulfonamide. In some embodiments, the composition comprises about 15% w/w to about 25% w/w of N-(1′,2-dihydroxy-1,2′-binaphthalen-4′-yl)-4-methoxybenzenesulfonamide. In some embodiments, the composition comprises about 20% w/w to about 25% w/w of N-(1′,2-dihydroxy-1,2′-binaphthalen-4′-yl)-4-methoxybenzenesulfonamide. In some embodiments, the composition comprises about 25% w/w to about 30% w/w of N-(1′,2-dihydroxy-1,2′-binaphthalen-4′-yl)-4-methoxy benzenesulfonamide.

In some embodiments, the composition comprises about 5% w/w of N-(1′,2-dihydroxy-1,2′-binaphthalen-4′-yl)-4-methoxybenzenesulfonamide. In some embodiments, the composition comprises about 10% w/w of N-(1′,2-dihydroxy-1,2′-binaphthalen-4′-yl)-4-methoxybenzenesulfonamide. In some embodiments, the composition comprises about 15% w/w of N-(1′,2-dihydroxy-1,2′-binaphthalen-4′-yl)-4-methoxybenzenesulfonamide. In some embodiments, the composition comprises about 20% w/w of N-(1′,2-dihydroxy-1,2′-binaphthalen-4′-yl)-4-methoxybenzenesulfonamide. In some embodiments, the composition comprises about 25% w/w of N-(1′,2-dihydroxy-1,2′-binaphthalen-4′-yl)-4-methoxybenzenesulfonamide. In some embodiments, the composition comprises about 30% w/w of N-(1′,2-dihydroxy-1,2′-binaphthalen-4′-yl)-4-methoxybenzenesulfonamide. In some embodiments, the composition comprises about 35% w/w of N-(1′,2-dihydroxy-1,2′-binaphthalen-4′-yl)-4-methoxybenzenesulfonamide. In some embodiments, the composition comprises about 40% w/w of N-(1′,2-dihydroxy-1,2′-binaphthalen-4′-yl)-4-methoxybenzenesulfonamide.

In some embodiments, the composition comprises about 5% w/w to about 50% w/w of the polymer excipient. In some embodiments, the composition comprises about 5% w/w to about 40% w/w of the polymer excipient. In some embodiments, the composition comprises about 10% w/w to about 40% w/w of the polymer excipient. In some embodiments, the composition comprises about 15% w/w to about 40% w/w of the polymer excipient. In some embodiments, the composition comprises about 20% w/w to about 40% w/w of the polymer excipient. In some embodiments, the composition comprises about 5% w/w to about 30% w/w of the polymer excipient. In some embodiments, the composition comprises about 10% w/w to about 30% w/w of the polymer excipient. In some embodiments, the composition comprises about 20% N w/w to about 35% w/w of the polymer excipient. In some embodiments, the composition comprises about 25% w/w to about 35% w/w of the polymer excipient. In some embodiments, the composition comprises about 15% w/w to about 30% w/w of the polymer excipient. In some embodiments, the composition comprises about 15% w/w to about 25% w/w of the polymer excipient. In some embodiments, the composition comprises about 20% w/w to about 25% w/w of the polymer excipient. In some embodiments, the composition comprises about 25% w/w to about 30% w/w of the polymer excipient.

In some embodiments, the composition comprises about 5% w/w of the polymer excipient. In some embodiments, the composition comprises about 10% w/w of the polymer excipient. In some embodiments, the composition comprises about 15% w/w of the polymer excipient. In some embodiments, the composition comprises about 20% w/w of the polymer excipient. In some embodiments, the composition comprises about 25% w/w of the polymer excipient. In some embodiments, the composition comprises about 30% w/w of the polymer excipient. In some embodiments, the composition comprises about 35% w/w of the polymer excipient. In some embodiments, the composition comprises about 40% w/w of the polymer excipient.

In some embodiments, the composition comprises 1:1 in w/w % of N-(1′,2-dihydroxy-1,2′-binaphthalen-4′-yl)-4-methoxy benzenesulfonamide to the polymer excipient. In some embodiments, the composition comprises 1:1.5 in w/w % of N-(1′,2-dihydroxy-1,2′-binaphthalen-4′-yl)-4-methoxybenzenesulfonamide to the polymer excipient. In some embodiments, the composition comprises 1.5:1 in w/w % of N-(1′,2-dihydroxy-1,2′-binaphthalen-4′-yl)-4-methoxybenzenesulfonamide to the polymer excipient. In some embodiments, the composition comprises 1:2 in w/w % of N-(1′,2-dihydroxy-1,2′-binaphthalen-4′-yl)-4-methoxybenzenesulfonamide to the polymer excipient. In some embodiments, the composition comprises 2:1 in w/w % of N-(1′,2-dihydroxy-1,2′-binaphthalen-4′-yl)-4-methoxybenzenesulfonamide to the polymer excipient.

Examples of crystallization inhibiting polymers include, but are not limited to, celluloses and derivatives thereof (e.g., microcrystalline cellulose, carboxymethylcellulose, hydroxypropyl methylcellulose (HPMC) or hypromellose, hydroxypropyl methylcellulose acetate succinate (HPMCAS) or hypromellose acetate succinate (e.g., HPMCAS-LG, HPMCAS-MG, HPMCAS-HG, HPMCAS-LF, HPMCAS-LMP), HPMC phthalate (HPMCP) (e.g., HPMCP-HP55, HPMCP-HP55S), Methocel™ (e.g., Methocel™ E3 (e.g., Methocel™ E3LV)), hydroxypropyl cellulose, hydroxyethylcellulose, ethylcellulose, and cellulose acetate phthalate).

In some embodiments, the crystallization inhibiting polymer is a methyl cellulose or HPMC. In some embodiments, the crystallization inhibiting polymer is HPMC. In some embodiments, the crystallization inhibiting polymer is HPMCAS (e.g., HPMCAS-LF, HPMCAS-LMP).

In some embodiments, the composition comprises about 1% w/w to about 40% w/w of the crystallization inhibiting polymer. In some embodiments, the composition comprises about 5% w/w to about 40% w/w of the crystallization inhibiting polymer. In some embodiments, the composition comprises about 10% w/w to about 40% w/w of the crystallization inhibiting polymer. In some embodiments, the composition comprises about 5% w/w to about 30% w/w of the crystallization inhibiting polymer. In some embodiments, the composition comprises about 15% N w/w to about 35% w/w of the crystallization inhibiting polymer. In some embodiments, the composition comprises about 10% w/w to about 30% w/w of the crystallization inhibiting polymer. In some embodiments, the composition comprises about 15% w/w to about 40% w/w of the crystallization inhibiting polymer. In some embodiments, the composition comprises about 20% w/w to about 40% w/w of the crystallization inhibiting polymer. In some embodiments, the composition comprises about 5% w/w to about 30% w/w of the crystallization inhibiting polymer. In some embodiments, the composition comprises about 10% w/w to about 30% w/w of the crystallization inhibiting polymer. In some embodiments, the composition comprises about 15% w/w to about 30% w/w of the crystallization inhibiting polymer. In some embodiments, the composition comprises about 15% w/w to about 25% w/w of the crystallization inhibiting polymer. In some embodiments, the composition comprises about 20% w/w to about 30% w/w of the crystallization inhibiting polymer.

In some embodiments, the composition comprises about 1% w/w of the crystallization inhibiting polymer. In some embodiments, the composition comprises about 2% w/w of the crystallization inhibiting polymer. In some embodiments, the composition comprises about 3% w/w of the crystallization inhibiting polymer. In some embodiments, the composition comprises about 4% w/w of the crystallization inhibiting polymer. In some embodiments, the composition comprises about 5% w/w of the crystallization inhibiting polymer. In some embodiments, the composition comprises about 10% w/w of the crystallization inhibiting polymer. In some embodiments, the composition comprises about 15% w/w of the crystallization inhibiting polymer. In some embodiments, the composition comprises about 20% w/w of the crystallization inhibiting polymer. In some embodiments, the composition comprises about 25% w/w of the crystallization inhibiting polymer. In some embodiments, the composition comprises about 30% w/w of the crystallization inhibiting polymer. In some embodiments, the composition comprises about 35% w/w of the crystallization inhibiting polymer. In some embodiments, the composition comprises about 40% w/w of the crystallization inhibiting polymer.

In some embodiments, when the selected crystallization inhibiting polymer for the composition is the same as the polymer excipient, the composition comprises about 20% to about 60% w/w of the crystallization inhibiting polymer/polymer excipient. In some embodiments, when the crystallization inhibiting polymer for the composition is the same as the polymer excipient, the composition comprises about 20% to about 50% w/w of the crystallization inhibiting polymer/polymer excipient. In some embodiments, when the crystallization inhibiting polymer for the composition is the same as the polymer excipient, the composition comprises about 30% to about 60% w/w of the crystallization inhibiting polymer/polymer excipient. In some embodiments, when the crystallization inhibiting polymer for the composition is the same as the polymer excipient, the composition comprises about 30% to about 50% w/w of the crystallization inhibiting polymer/polymer excipient. In some embodiments, when the crystallization inhibiting polymer for the composition is the same as the polymer excipient, the composition comprises about 40% to about 60% w/w of the crystallization inhibiting polymer/polymer excipient. In some embodiments, when the crystallization inhibiting polymer for the composition is the same as the polymer excipient, the composition comprises about 40% to about 50% w/w of the crystallization inhibiting polymer/polymer excipient.

In some embodiments, when the crystallization inhibiting polymer for the composition is the same as the polymer excipient, the composition comprises about 20% w/w of the crystallization inhibiting polymer/polymer excipient. In some embodiments, when the crystallization inhibiting polymer for the composition is the same as the polymer excipient, the composition comprises about 30% w/w of the crystallization inhibiting polymer/polymer excipient. In some embodiments, when the crystallization inhibiting polymer for the composition is the same as the polymer excipient, the composition comprises about 40% w/w of the crystallization inhibiting polymer/polymer excipient. In some embodiments, when the crystallization inhibiting polymer for the composition is the same as the polymer excipient, the composition comprises about 50% w/w of the crystallization inhibiting polymer/polymer excipient. In some embodiments, when the crystallization inhibiting polymer for the composition is the same as the polymer excipient, the composition comprises about 60% w/w of the crystallization inhibiting polymer/polymer excipient.

In some embodiments, the composition comprises at least 100 mg of a N-(1′,2-dihydroxy-1,2′-binaphthalen-4′-yl)-4-methoxybenzenesulfonamide. In some embodiments, the composition comprises at least 110 mg of a N-(1′,2-dihydroxy-1,2′-binaphthalen-4′-yl)-4-methoxybenzenesulfonamide. In some embodiments, the composition comprises at least 120 mg of a N-(1′,2-dihydroxy-1,2′-binaphthalen-4′-yl)-4-methoxybenzenesulfonamide. In some embodiments, the composition comprises at least 130 mg of a N-(1′,2-dihydroxy-1,2′-binaphthalen-4′-yl)-4-methoxybenzenesulfonamide. In some embodiments, the composition comprises at least 140 mg of a N-(1′,2-dihydroxy-1,2′-binaphthalen-4′-yl)-4-methoxybenzenesulfonamide. In some embodiments, the composition comprises at least 150 mg of a N-(1′,2-dihydroxy-1,2′-binaphthalen-4′-yl)-4-methoxybenzenesulfonamide. In some embodiments, the composition comprises at least 160 mg of a N-(1′,2-dihydroxy-1,2′-binaphthalen-4′-yl)-4-methoxybenzenesulfonamide. In some embodiments, the composition comprises at least 170 mg of a N-(1′,2-dihydroxy-1,2′-binaphthalen-4′-yl)-4-methoxybenzenesulfonamide. In some embodiments, the composition comprises at least 180 mg of a N-(1′,2-dihydroxy-1,2′-binaphthalen-4′-yl)-4-methoxybenzenesulfonamide. In some embodiments, the composition comprises at least 190 mg of a N-(1′,2-dihydroxy-1,2′-binaphthalen-4′-yl)-4-methoxybenzenesulfonamide. In some embodiments, the composition comprises at least 200 mg of a N-(1′,2-dihydroxy-1,2′-binaphthalen-4′-yl)-4-methoxybenzenesulfonamide. In some embodiments, the composition comprises at least 300 mg of a N-(1′,2-dihydroxy-1,2′-binaphthalen-4′-yl)-4-methoxybenzenesulfonamide. In some embodiments, the composition comprises at least 400 mg of a N-(1′,2-dihydroxy-1,2′-binaphthalen-4′-yl)-4-methoxybenzenesulfonamide. In some embodiments, the composition comprises at least 500 mg of a N-(1′,2-dihydroxy-1,2′-binaphthalen-4′-yl)-4-methoxybenzenesulfonamide.

In some embodiments, the composition comprises about 200 mg of a N-(1′,2-dihydroxy-1,2′-binaphthalen-4′-yl)-4-methoxybenzenesulfonamide. In some embodiments, the composition comprises about 250 mg of a N-(1′,2-dihydroxy-1,2′-binaphthalen-4′-yl)-4-methoxybenzenesulfonamide. In some embodiments, the composition comprises about 300 mg of a N-(1′,2-dihydroxy-1,2′-binaphthalen-4′-yl)-4-methoxybenzenesulfonamide. In some embodiments, the composition comprises about 350 mg of a N-(1′,2-dihydroxy-1,2′-binaphthalen-4′-yl)-4-methoxybenzenesulfonamide. In some embodiments, the composition comprises about 400 mg of a N-(1′,2-dihydroxy-1,2′-binaphthalen-4′-yl)-4-methoxybenzenesulfonamide. In some embodiments, the composition comprises about 450 mg of a N-(1′,2-dihydroxy-1,2′-binaphthalen-4′-yl)-4-methoxybenzenesulfonamide. In some embodiments, the composition comprises about 500 mg of a N-(1′,2-dihydroxy-1,2′-binaphthalen-4′-yl)-4-methoxybenzenesulfonamide.

In some embodiments, the composition further comprises at least one additional component. Said component can be intra-granular or extra-granular components that improve processability of the tablet manufacture (e.g., processability of roller compaction, tablet compression and film coating). The additional components can also impart good powder flow and compression characteristics to the material being compressed. Desirable characteristics of the additional components can include high-compressibility so as to allow for strong tablets to be made at low compression forces; good powder flow properties that can improve the powder flow of other excipients in the composition; and cohesiveness, for example to prevent a tablet from crumbling during processing, shipping, and handling. Other components which give physical characteristics to a finished tablet are coloring and flavoring agents (e.g., in the case of chewable tablets). Examples of additional components are described, for example, in the Handbook of Pharmaceutical Excipients (5^{th} edition), Edited by Raymond C Rowe. Paul J. Sheskey, and Sian C. Owen: Publisher: Pharmaceutical Press.

In some embodiments, the additional components include, but are not limited to, diluents, binders, fillers, disintegrants, surfactants, lubricants, flavoring agents, and colors. The additional component can serve multiple functions. For example, a diluent, in some embodiments, also serve as a filler. As another example, a surfactant, in some embodiments, also serve as a lubricant.

In some embodiments, diluents or fillers are added, for example, to increase the bulk weight of the blend resulting in a practical size for compression. Diluents or fillers that, in some embodiments, are used include one or more of calcium salts such as calcium phosphate dibasic and sugars such as lactose, sucrose, dextrose, microcrystalline cellulose, mannitol, and maltodextrin. Examples of pharmaceutically acceptable fillers and pharmaceutically acceptable diluents include, but are not limited to, confectioner's sugar, compressible sugar, dextrates, dextrin, dextrose, lactose, mannitol, microcrystalline cellulose, powdered cellulose, sorbitol, sucrose and talc. In some embodiments, the diluent or filler is microcrystalline cellulose, which can be manufactured by the controlled hydrolysis of alpha-cellulose. In some embodiments, suitable microcrystalline cellulose has an average particle size of from about 20 nm to about 200 nm. Suitable microcrystalline cellulose includes Avicel PH 101, Avicel PH 102, Avicel PH 103, Avicel PH 105 and Avicel PH 200. In some embodiments, the diluent or filler is silicified microcrystalline cellulose, such as ProSolv® SMCC 50 or ProSolv® SMCC HD 90. In some embodiments, the diluent or filler is lactose. In some embodiments, the diluent or filler is a mixture of two or more diluents or fillers. In some embodiments, the diluent or filler is microcrystalline cellulose, silicified microcrystalline cellulose, or powdered cellulose, or a mixture thereof.

In some embodiments, the amount of diluent or filler employed in the composition is from about 5% to about 50% w/w. In some embodiments, the amount of diluent or filler employed in the composition is from about 5% to about 40% w/w. In some embodiments, the amount of diluent or filler employed in the composition is from about 5% to about 30% w/w. In some embodiments, the amount of diluent or filler employed in the composition is from about 5% to about 25% w/w. In some embodiments, the amount of diluent or filler employed in the composition is from about 5% to about 20% w/w. In some embodiments, the amount of diluent or filler employed in the composition is from about 5% to about 15% w/w. In some embodiments, the amount of diluent or filler employed in the composition is from about 5% to about 10% w/w. In some embodiments, the amount of diluent or filler employed in the composition is from about 10% to about 50% w/w. In some embodiments, the amount of diluent or filler employed in the composition is from about 10% to about 40% w/w. In some embodiments, the amount of diluent or filler employed in the composition is from about 10% to about 30% w/w. In some embodiments, the amount of diluent or filler employed in the composition is from about 10% to about 20% w/w. In some embodiments, the amount of diluent or filler employed in the composition is from about 15% to about 40% w/w. In some embodiments, the amount of diluent or filler employed in the composition is from about 15% to about 35% w/w. In some embodiments, the amount of diluent or filler employed in the composition is from about 15% to about 30% w/w. In some embodiments, the amount of diluent or filler employed in the composition is from about 15% to about 20% w/w. In some embodiments, the amount of diluent or filler employed in the composition is from about 20% to about 50% w/w. In some embodiments, the amount of diluent or filler employed in the composition is from about 20% to about 40% w/w. In some embodiments, the amount of diluent or filler employed in the composition is from about 20% to about 35% w/w. In some embodiments, the amount of diluent or filler employed in the composition is from about 5% to about 20% w/w. In some embodiments, the amount of diluent or filler employed in the composition is from about 20% to about 30% w/w. In some embodiments, the amount of diluent or filler employed in the composition is about 5% w/w. In some embodiments, the amount of diluent or filler employed in the composition is about 10% w/w. In some embodiments, the amount of diluent or filler employed in the composition is about 15% w/w. In some embodiments, the amount of diluent or filler employed in the composition is about 20% w/w. In some embodiments, the amount of diluent or filler employed in the composition is about 25% w/w. In some embodiments, the amount of diluent or filler employed in the composition is about 30% w/w. In some embodiments, the amount of diluent or filler employed in the composition is about 35% w/w. In some embodiments, the amount of diluent or filler employed in the composition is about 40% w/w. In some embodiments, the amount of diluent or filler employed in the composition is about 45% w/w. In some embodiments, the amount of diluent or filler employed in the composition is about 50% w/w.

In some embodiments, the composition further includes a lubricant. Lubricants are typically added to prevent the tableting materials from clumping together and from stick to the tablet punches, minimize friction during tablet compression, and to allow for removal of the compressed tablet from the die. Such lubricants are included in the final tablet mix in amounts usually less than 5 by weight per weight of a composition. Examples of lubricants include, but are not limited to, colloidal silica, magnesium trisilicate, talc, magnesium carbonate, stearic acid, magnesium oxide, glycerylbehaptate, polyethylene glycol, ethylene oxide polymers, sodium lauryl sulphate, magnesium stearate, aluminum stearate, calcium stearate, sodium stearyl fumarate, stearic acid, magnesium lauryl stearate, and mixtures of magnesium stearate with sodium lauryl sulphate. In some embodiments, the lubricant is sodium lauryl sulfate or sodium stearyl fumarate, or a mixture thereof. In some embodiments, the lubricant is a mixture of two or more lubricants.

In some embodiments, the amount of lubricant employed in the composition is from about 0.01 to about 5.0% w/w. In some embodiments, the amount of lubricant employed in the composition is from about 0.01 to about 4.0% w/w. In some embodiments, the amount of lubricant employed in the composition is from about 0.01 to about 3.0% w/w. In some embodiments, the amount of lubricant employed in the composition is from about 0.1 to about 3.0% w/w. In some embodiments, the amount of lubricant employed in the composition is from about 0.01 to about 2.0% w/w. In some embodiments, the amount of lubricant employed in the composition is from about 0.05 to about 2.0% w/w. In some embodiments, the amount of lubricant employed in the composition is from about 0.1 to about 2.0% w/w. In some embodiments, the amount of lubricant employed in the composition is from about 0.01 to about 1.5% w/w. In some embodiments, the amount of lubricant employed in the composition is from about 0.05 to about 1.5% w/w. In some embodiments, the amount of lubricant employed in the composition is from about 0.1 to about 1.5% w/w. In some embodiments, the amount of lubricant employed in the composition is from about 0.5 to about 4.0% w/w. In some embodiments, the amount of lubricant employed in the composition is from about 0.5 to about 3.5% w/w. In some embodiments, the amount of lubricant employed in the composition is from about 0.5 to about 3.0 w/w. In some embodiments, the amount of lubricant employed in the composition is from about 0.5 to about 2.5% w/w. In some embodiments, the amount of lubricant employed in the composition is from about 0.5 to about 2.0% w/w. In some embodiments, the amount of lubricant employed in the composition is from about 0.5 to 1.5% w/w. In some embodiments, the amount of lubricant employed in the composition is from about 1.0 to about 3.0% w/w. In some embodiments, the amount of lubricant employed in the composition is from about 1.0 to about 2.5% w/w. In some embodiments, the amount of lubricant employed in the composition is from about 1.0 to about 2.0% w/w. In some embodiments, the amount of lubricant employed in the composition is from about 1.5 to about 2.0% w/w. In some embodiments, the amount of lubricant employed in the composition is from about 0.5 to 1.0% w/w. In some embodiments, the amount of lubricant employed in the composition is about 0.1% w/w. In some embodiments, the amount of lubricant employed in the composition is about 0.2% w/w. In some embodiments, the amount of lubricant employed in the composition is about 0.3% w/w. In some embodiments, the amount of lubricant employed in the composition is about 0.4% w/w. In some embodiments, the amount of lubricant employed in the composition is about 0.5% w/w. In some embodiments, the amount of lubricant employed in the composition is about 0.6% w/w. In some embodiments, the amount of lubricant employed in the composition is about 0.7% w/w. In some embodiments, the amount of lubricant employed in the composition is about 0.8% w/w. In some embodiments, the amount of lubricant employed in the composition is about 0.9% w/w. In some embodiments, the amount of lubricant employed in the composition is about 1.0% w/w. In some embodiments, the amount of lubricant employed in the composition is about 1.5% w/w. In some embodiments, the amount of lubricant employed in the composition is about 1.8% w/w. In some embodiments, the amount of lubricant employed in the composition is about 2.0% w/w. In some embodiments, the amount of lubricant employed in the composition is about 2.5% w/w.

In some embodiments, glidants are added, for example, to improve tablet powder's flowability by reducing interparticle friction and cohesion. Examples of glidants include magnesium stearate, magnesium carbonate, silica (e.g., colloidal silicon dioxide (such as the grades sold as Aerosil®)), starch and talc. Glidants, in some embodiments, are present in the composition at an amount of from 0.01 to about 5% w/w. In some embodiments, the amount of glidant employed in the composition is from about 0.01 to about 4.0% w/w. In some embodiments, the amount of glidant employed in the composition is from about 0.01 to about 3.0% w/w. In some embodiments, the amount of glidant employed in the composition is from about 0.01 to about 2.0% w/w. In some embodiments, the amount of glidant employed in the composition is from about 0.05 to about 2.0% w/w. In some embodiments, the amount of glidant employed in the composition is from about 0.1 to about 2.0% w/w. In some embodiments, the amount of glidant employed in the composition is from about 0.01 to about 1.5% w/w. In some embodiments, the amount of glidant employed in the composition is from about 0.05 to about 1.5% w/w. In some embodiments, the amount of glidant employed in the composition is from about 0.1 to about 1.5% w/w. In some embodiments, the amount of glidant employed in the composition is from about 0.5 to 1.5% w/w. In some embodiments, the amount of glidant employed in the composition is from about 0.5 to 1.0% w/w. In some embodiments, the amount of glidant employed in the composition is about 0.1% w/w. In some embodiments, the amount of glidant employed in the composition is about 0.2% w/w. In some embodiments, the amount of glidant employed in the composition is about 0.3% w/w. In some embodiments, the amount of glidant employed in the composition is about 0.4% w/w. In some embodiments, the amount of glidant employed in the composition is about 0.5% w/w. In some embodiments, the amount of glidant employed in the composition is about 0.6% w/w. In some embodiments, the amount of glidant employed in the composition is about 0.7% w/w. In some embodiments, the amount of glidant employed in the composition is about 0.8% w/w. In some embodiments, the amount of glidant employed in the composition is about 0.9% w/w. In some embodiments, the amount of glidant employed in the composition is about 1.0% w/w. In some embodiments, the amount of glidant employed in the composition is about 2.0% w/w.

In some embodiments, a tablet disintegrant is present in the composition at an amount, for example, to expedite dissolution (e.g., increase the rate of tablet disintegration). Disintegrants are excipients which can oppose the physical forces of particle bonding in a tablet when the dosage form is placed in an aqueous environment. Disintegrants include starch derivatives and salts of carboxymethylcellulose. Examples of pharmaceutically acceptable disintegrants include, but are not limited to, starches, e.g., sodium starch glycolate, pregelatinized starch; clays; celluloses; alginates; gums; cross-linked polymers, e.g., cross-linked polyvinyl pyrrolidone (e.g., polyvinyl polypyrrolidone, PVPP, crospovidone, crospolividone), cross-linked calcium carboxymethylcellulose and cross-linked sodium carboxymethylcellulose (sodium croscarmellose); and polysaccharides. In some embodiments, the disintegrant is a mixture of two or more disintegrants.

Generally the amount of disintegrant can be from 0.1 to about 25% w/w of the composition. In some embodiments, the amount of disintegrant is from about 1% to about 15% w/w of the composition. In some embodiments, the amount of disintegrant is from about 1% to about 10% w/w of the composition. In some embodiments, the amount of disintegrant is from about 1% to about 8% w/w of the composition. In some embodiments, the amount of disintegrant is from about 5% to about 10% w/w of the composition. In some embodiments, the amount of disintegrant is from about 3% to about 10% w/w of the composition. In some embodiments, the amount of disintegrant is from about 3% to about 8% w/w of the composition. In some embodiments, the amount of disintegrant is from about 5% to about 10% w/w of the composition. In some embodiments, the amount of disintegrant is from about 5% to about 15% w/w of the composition. In some embodiments, the amount of disintegrant is from about 1% to about 5% w/w of the composition. In some embodiments, the amount of disintegrant is about 1% w/w of the composition. In some embodiments, the amount of disintegrant is about 2% w/w of the composition. In some embodiments, the amount of disintegrant is about 3% w/w of the composition. In some embodiments, the amount of disintegrant is about 4% w/w of the composition. In some embodiments, the amount of disintegrant is about 5% w/w. In some embodiments, the amount of disintegrant is about 6% w/w of the composition. In some embodiments, the amount of disintegrant is about 7% w/w of the composition. In some embodiments, the amount of disintegrant is about 8% w/w of the composition. In some embodiments, the amount of disintegrant is about 9% w/w of the composition. In some embodiments, the amount of disintegrant is about 10% w/w of the composition. In some embodiments, the amount of disintegrant is about 15% w/w of the composition.

In some embodiments, the methods described herein contemplate administering to the subject an oral dosage form comprising the composition as described herein in the form of a tablet.

In some embodiments, a composition is set forth as in Table C or Table D (e.g., wherein the total wt. % in the composition does not exceed 100%).

TABLE C
Exemplary Compositions

| | | | |
|---|---|---|---|
| | Composition 1 | Composition 2 | Composition 3 |
| Component | (w/w) | (w/w) | (w/w) |

Spray-Dried Dispersion - Solid Amorphous Dispersion

| | | | |
|---|---|---|---|
| TTI-101 | 10-30% | 15-25% | 22% |
| Polymer excipient | 10-30% | 15-25% | 22% |

Intra-granular Blend

| | | | |
|---|---|---|---|
| Diluent/Filler | 5-20% | 10-15% | 12% |
| Disintegrant | 0.5-5% | 1-5% | 3.2% |
| Surfactant/ | 0.1-2% | 0.5-1.0% | 0.8% |
| Lubricant | | | |
| CIP | 5-25% | 10-20% | 15% |
| CIP | 1-10% | 5-10% | 6% |

Extra-granular Blend

| | | | |
|---|---|---|---|
| Diluent/Filler | 5-25% | 10-20% | 15% |
| Disintegrant | 0.5-10% | 1-5% | 3% |
| Lubricant | 0.1-2% | 0.5-1.5% | 1% |

TABLE D
Exemplary Compositions

| | Composition 4 | Composition 5 | Composition 6 |
|---|---|---|---|
| Component | (w/w) | (w/w) | (w/w) |
| TTI-101 | 10-30% | 15-25% | 22% |
| Polymer excipient | 10-30% | 15-25% | 22% |
| Diluent/Filler | 15-40% | 20-35% | 27% |
| Disintegrant | 1-10% | 3-8% | 6% |
| Surfactant/Lubricant | 0.1-2% | 0.5-1.5% | 1% |
| CIP | 10-30% | 15-25% | 21% |
| Lubricant | 0.1-2% | 0.5-1.5% | 1% |

Tablets, in some embodiments, are plain, film, sugar coated, bisected, embossed, layered, and/or sustained-release. They can be made in a variety of sizes, shapes, and colors. Tablets, in some embodiments, are swallowed, chewed, or dissolved in the buccal cavity or beneath the tongue.

In some embodiments, the tablet is coated or otherwise compounded to provide a dosage form affording the advantage of prolonged action, protect tablet ingredients from deterioration, make large or unpleasant-tasking tablets easier to swallow, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

In some embodiments, the tablet is polymer-coated.

In some embodiments, the tablet is coated with Opadry® EZ white.

Components of an exemplary 200 mg tablet comprising TTI-101 is shown in Table E.

TABLE E
Example 200 mg TTI-101 Tablet Formulation

| | | | |
|---|---|---|---|
| | | % | Quantity |
| Component | Function | w/w | per Unit |

Spray-Dried Dispersion

| | | | |
|---|---|---|---|
| TTI-101 | Active ingredient | 22.28% | 200 mg |
| Methacrylic Acid and | Polymer | 22.28% | 200 mg |
| Ethyl Acrylate Copolymer | Excipient | | |

Intra-granular Blend

| | | | |
|---|---|---|---|
| Silicified Microcrystalline | Diluent/Filler | 12.15% | 109.1 mg |
| Cellulose | | | |
| Croscarmellose Sodium | Disintegrant | 3.24% | 29.1 mg |
| Sodium Lauryl Sulfate | Surfactant/ | 0.81% | 7.3 mg |
| | Lubricant | | |
| Hypromellose acetate | Crystallization | 14.58% | 130.9 mg |
| succinate LMP | inhibitor | | |
| Hypromellose acetate | Crystallization | 5.67% | 50.9 mg |
| succinate LF | inhibitor | | |

| | | |
|---|---|---|
| Total: | 81.00% | 727.3 mg |

Extra-granular Blend

| | | | |
|---|---|---|---|
| Silicified Microcrystalline | Diluent/Filler | 15.00% | 134.7 mg |
| Cellulose | | | |
| Croscarmellose Sodium | Disintegrant | 3.00% | 26.9 mg |
| Sodium Stearyl Fumarate | Lubricant | 1.00% | 9.0 mg |

| | | |
|---|---|---|
| Total: | 100.00% | 897.9 mg |

Coating

| | | | |
|---|---|---|---|
| Opadry EZ White | Coating film | 3.0% of | 26.9 mg |
| | | core | |

In some embodiments, the compositions described herein is administered as a suspension in a pharmaceutically acceptable carrier (e.g., after, optionally, crushing the compositions or solid oral dosage forms into fine powder for the suspension). In some embodiments, the suspension is administered orally. In some embodiments, the suspension is administered via a feeding tube (e.g., via percutaneous endoscopic gastronomy (PEG) tube or G tube). PEG allows for a feeding tube to be place through the abdominal wall and into the stomach, allowing the composition to be put directly into the stomach, bypassing the mouth and esophagus.

### EXAMPLES

### Example 1. Safety and Efficacy of TTI-101 in Non-Viral Liver Cancer

This study evaluates the safety and efficacy of TTI-101 in advanced solid tumors. TTI-101 was administered twice daily at doses of 1.6 (Cohort 1; n=4), 3.2 (Cohort 2; n=3), or 6.4 mg/kg/dose (Cohort 3; n=8) of a Labrasol/PEG400 formulation (60:40% v/v) or 6.4 mg/kg/dose (Cohort 3a: n=37) or 12.8 mg/kg/dose (Cohort 4; n=10) of oral capsule formulation (composition comprising TTI-101 at a concentration of 92 mg/g in a mixture of Kolliphor RH 40 (PEG-40 hydrogenated castor oil). PEG600. Polysorbate 20, Labrasol, and citric acid). TTI-101 was administered twice daily at 6.4 mg/kg/dose (Cohort 5; n=7) of oral tablet formulation (comprising 200 mg of TTI-101).

Preliminary data demonstrates among 15 evaluable participants with HCC treated with TTI-101 as monotherapy, after two 28-day cycles of TTI-101 treatment, 3 radiologically confirmed partial responses occurred. These patients had failed all prior lines of therapy (median number of prior therapies=3). Furthermore, a disease control rate (defined as the proportion of patients in whom the best overall response is determined as complete response, partial response or stable disease according to RECIST v1.1) of 60% has been observed in the 15 heavily pre-treated HCC patients (9/15), which is similar to that reported with sorafenib in first-line therapy.

Table 1 below shows the best response by cohort among HCC participants at first data point:

TABLE 1

| | | HCC | HCC |
|---|---|---|---|
| HCC Patients | HCC | (non-viral) | (viral) |
| Partial Response | 3 | 3 | 0 |
| Stable Disease | 6 | 1 | 5 |
| Progressive Disease | 6 | 3 | 3 |
| Totals | 15 | 7 | 8 |
| Disease Control Rate | 60% | 57% | 63% |

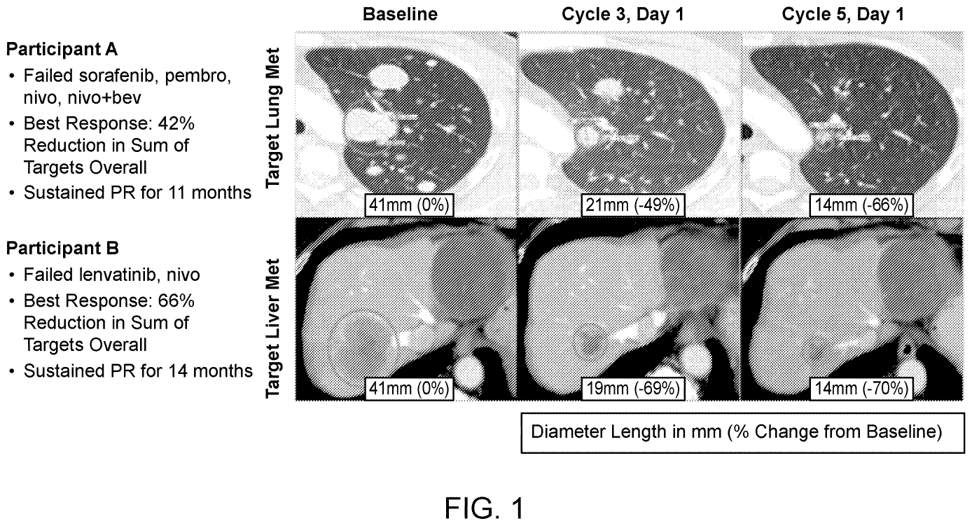

Seven of the 15 participants with HCC had non-viral etiologies. The 3 participants demonstrating partial responses had non-viral etiologies and are described in more detail below. Available images from the first two responders are highlighted in FIG. 1.

Participant A (top panel in FIG. 1) had previously failed treatment with sorafenib, pembrolizumab, nivolumab, and nivolumab+bevacizumab before initiating treatment with 6.4 mg/kg/day of TTI-101. Their best response was a 42% reduction in the sum of overall RECIST targets. They sustained the partial response for 11 months.

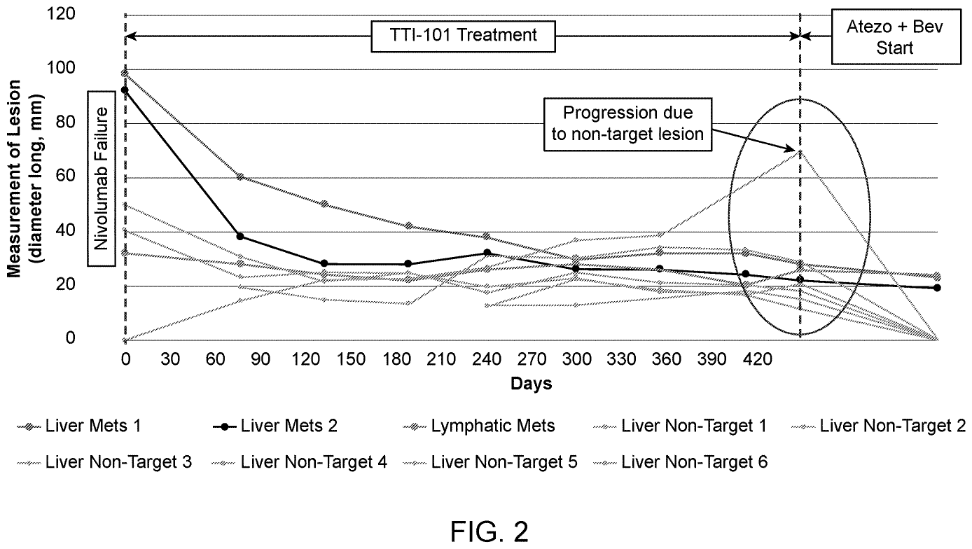

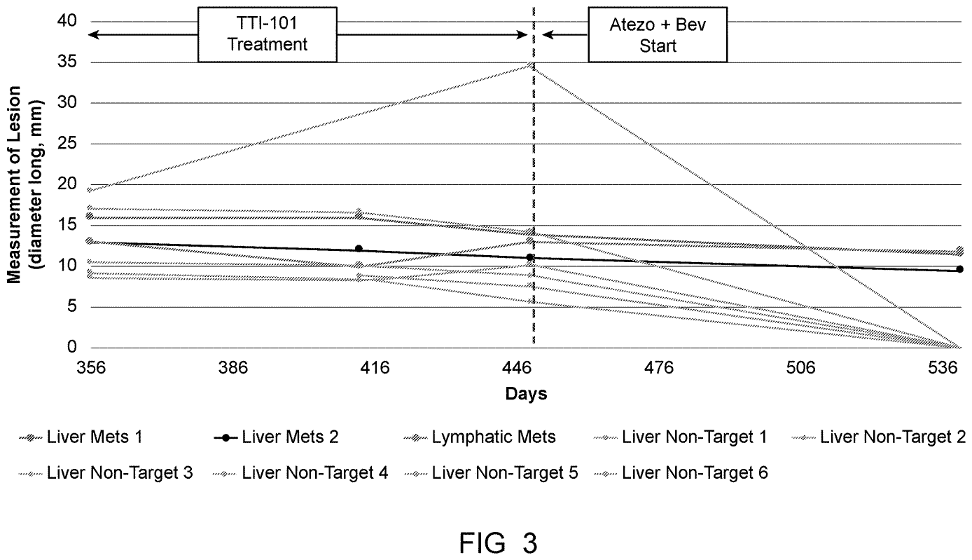

Participant B (bottom panel in FIG. 1) previously failed treatment with lenvatinib and nivolumab before initiating treatment with 12.8 mg/kg/day of TTI-101. Their best response was a 66% reduction in the sum of overall RECIST targets (FIG. 2). They sustained the partial response for 14 months, after which time Participant B demonstrated disease progression and discontinued treatment with TTI-101. Participant B was subsequently treated with atezolizumab+bevacizumab within 30 days of discontinuation of TTI-101, and after 2 months of treatment demonstrated a new response, with decreases in target and nontarget lesions, suggesting a use of TTI-101 for resensitizing the tumor to immune checkpoint inhibitor (ICI) therapy (FIG. 3).

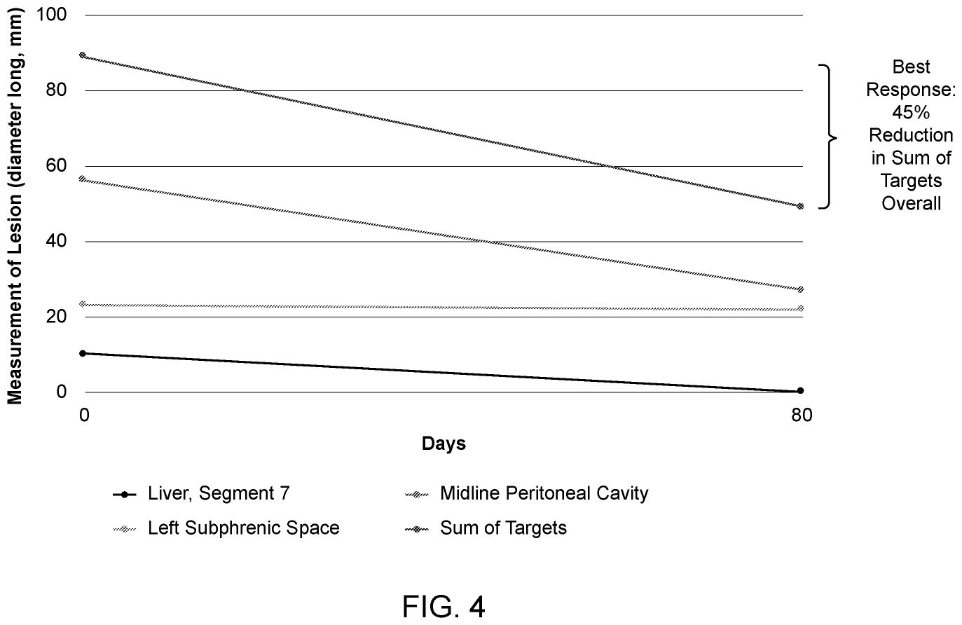

Participant C previously failed atezolizumab+bevacizumab before initiating treatment with 12.8 mg/kg/day of TTI-101. Their best response was a 45% reduction in the sum of overall RECIST targets (FIG. 4)

In the phase 1b/2 study, the safety and efficacy of treatment with TTI-101 will be evaluated as monotherapy (cohort A), in combination with pembrolizumab (cohort B), or in combination with atezolizumab+bevacizumab (cohort C), in male and female participants with locally advanced or metastatic, and unresectable HCC. In cohort A, TTI-101 will be administered as a single agent in participants who have recently demonstrated objective progression on up to 3 prior lines of systemic antitumor drug therapy. In cohort B, TTI-101 will be administered in combination with pembrolizumab in participants who have recently demonstrated objective progression following at least 3 months of first-line anti-PD-1 or anti-PD-L1 monotherapy or combination therapy. In cohort C, TTI-101 will be administered in combination with atezolizumab and bevacizumab in participants who were treatment-naïve. 50% of the participants to be enrolled in each cohort in phase 2 will be of non-viral etiology.

TTI-101 will be administered as a 200 mg spray-dried dispersion (SDD) tablets. Exemplary doses are: 800 mg/day (e.g., 4 tablets/day, taken as 2 tablets am/2 tablets pm orally), 1200 mg/day (e.g., 6 tablets/day, taken as 3 tablets am/3 tablets pm orally), and 1600 mg/day (e.g., 8 tablets/day, taken as 4 tablets am/4 tablets pm orally). In cohort B, pembrolizumab will be administered as an IV infusion, e.g., 400 mg every 6 weeks or 200 mg every 3 weeks (physician's choice). In cohort C, atezolizumab will be administered as sequential IV infusion, e.g., 1200 mg atezolizumab followed by 15 mg/kg bevacizumab on the same day every 3 weeks. If a dose-limiting toxicity (DLT) is observed in ≥2 participants at the 800 mg/day dose in phase Tb, the dose will be de-escalated to 400 mg/day.

1. A compound of Formula (I): 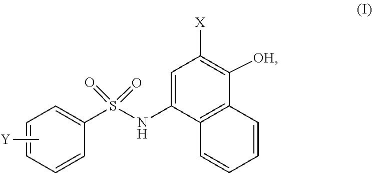
or a pharmaceutically acceptable salt thereof, for use in a method of treating a subject diagnosed with a non-viral liver cancer, the method comprising administering to the subject an effective amount of the compound of Formula (I), or a pharmaceutically acceptable salt thereof, wherein
X is selected from the group consisting of hydrogen, phenylsulfanyl, hydroxy-naphthalenyl, quinolin-8-ylsulfanyl, triazol-3-yl sulfanyl, and benzothiazol-2-ylsulfanyl; and
Y is selected from the group consisting of hydrogen, methyl, chloro, bromo, methoxy, ethoxy, tert-butyl, nitro, methyl ester, acetamide, 1,4 dioxine, fluoro, trifluoro methoxy, acetyl, trifluoro methyl, propyl, cyclohexene, methoxy-phenoxy, chloro phenoxy, tolyloxy, and phenoxy.

2. The compound for use of claim 1, wherein the compound of Formula (I) is: 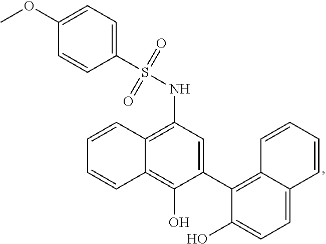 [SMILES: COc1ccc(S(=O)(=O)Nc2cc(-c3c(O)ccc4ccccc34)c(O)c3ccccc23)cc1]
or a pharmaceutically acceptable salt thereof.

3. The compound for use of claim 1, wherein the non-viral liver cancer is hepatocellular carcinoma, fibrolamellar carcinoma, bile duct cancer, angiosarcoma, or hepatoblastoma.

4. The compound for use of claim 1, wherein the non-viral liver cancer is hepatocellular carcinoma.

5. The compound for use of claim 1, wherein the subject has alcoholic liver disease, alcoholic cirrhosis, autoimmune disease, metabolic disease, alcoholic steatohepatitis, non-alcoholic fatty liver disease, non-alcoholic steatohepatitis, diabetes, obesity, iron overload syndrome, autoimmune hepatitis, primary biliary cholangitis, cryptogenic liver disease, alcohol use, tobacco use, oral contraceptive use, pesticide exposure, aflatoxin exposure, or betel quid chewing.

6. The compound for use of claim 1, wherein the subject is negative for hepatitis B virus.

7. The compound for use of claim 1, wherein the subject is negative for hepatitis B surface antigen.

8. The compound for use of claim 1, wherein the subject is negative for hepatitis C virus.

9. The compound for use of claim 1, wherein the subject is negative for anti-HCV antibody.

10. The compound for use of claim 1, wherein the subject has been administered one or more prior cancer therapy.

11. The compound for use of claim 10, wherein the prior cancer therapy is sorafenib, pembrolizumab, nivolumab, lenvatinib, bevacizumab, atezolizumab, ramucirumab, cabozantinib, infigratinib, pemigatinib, regorafenib, tremelimumab, or durvalumab, or pharmaceutically acceptable salts thereof, or any combination thereof.

12. The compound for use of claim 10, wherein the prior cancer therapy is sorafenib, pembrolizumab, nivolumab, lenvatinib, or bevacizumab.

13. The compound for use of claim 1, wherein the non-viral liver cancer has progressed from, or the subject was intolerant to, a prior first line cancer therapy.

14. The compound for use of claim 1, wherein the non-viral liver cancer has progressed from, or the subject was intolerant to, a prior second line cancer therapy.

15. The compound for use of claim 1, wherein the non-viral liver cancer has progressed from, or the subject was intolerant to, a prior third line cancer therapy.

16. The compound for use of claim 1, wherein about 1 mg/kg/day to about 50 mg/kg/day of the compound of Formula (I), or a pharmaceutically acceptable salt thereof, is administered to the subject.

17. The compound for use of claim 16, wherein about 5 mg/kg/day to about 30 mg/kg/day of the compound of Formula (I), or a pharmaceutically acceptable salt thereof, is administered to the subject.

18. The compound for use of claim 16, wherein about 5 mg/kg/day to about 8 mg/kg/day of the compound of Formula (I), or a pharmaceutically acceptable salt thereof, is administered to the subject.

19. The compound for use of claim 16, wherein about 10 mg/kg/day to about 15 mg/kg/day of the compound of Formula (I), or a pharmaceutically acceptable salt thereof, is administered to the subject.

20. The compound for use of claim 16, wherein about 20 mg/kg/day to about 30 mg/kg/day of the compound of Formula (I), or a pharmaceutically acceptable salt thereof, is administered to the subject.

21. The compound for use of claim 1, wherein about 0.5 mg/kg/dose to about 25 mg/kg/dose of the compound of Formula (I), or a pharmaceutically acceptable salt thereof, is administered to the subject.

22. The compound for use of claim 21, wherein about 2.5 mg/kg/dose to about 15 mg/kg/dose of the compound of Formula (I), or a pharmaceutically acceptable salt thereof, is administered to the subject.

23. The compound for use of claim 21, wherein about 2.5 mg/kg/day to about 4 mg/kg/day of the compound of Formula (I), or a pharmaceutically acceptable salt thereof, is administered to the subject.

24. The compound for use of claim 21, wherein about 5 mg/kg/day to about 7.5 mg/kg/day of the compound of Formula (I), or a pharmaceutically acceptable salt thereof, is administered to the subject.

25. The compound for use of claim 21, wherein about 10 mg/kg/day to about 15 mg/kg/day of the compound of Formula (I), or a pharmaceutically acceptable salt thereof, is administered to the subject.

26. The compound for use of any one of claims 21-25, wherein the subject is dosed twice daily of the compound of Formula (I), or a pharmaceutically acceptable salt thereof.

27. The compound for use of claim 1, wherein the subject is dosed twice daily of the compound of Formula (I), or a pharmaceutically acceptable salt thereof, at about 1.6 mg/kg/dose.

28. The compound for use of claim 1, wherein the subject is dosed twice daily of the compound of Formula (I), or a pharmaceutically acceptable salt thereof, at about 3.2 mg/kg/dose.

29. The compound for use of claim 1, wherein the subject is dosed twice daily of the compound of Formula (I), or a pharmaceutically acceptable salt thereof, at about 6.4 mg/kg/dose.

30. The compound for use of claim 1, wherein the subject is dosed twice daily of the compound of Formula (I), or a pharmaceutically acceptable salt thereof, at about 12.8 mg/kg/dose.

31. The compound for use of claim 1, wherein about 200 mg to about 2000 mg of the compound of Formula (I), or a pharmaceutically acceptable salt thereof, is administered to the subject per day.

32. The compound for use of claim 31, wherein about 400 mg of the compound of Formula (I), or a pharmaceutically acceptable salt thereof, is administered to the subject per day.

33. The compound for use of claim 31, wherein about 800 mg of the compound of Formula (I), or a pharmaceutically acceptable salt thereof, is administered to the subject per day.

34. The compound for use of claim 31, wherein about 800 mg of the compound of Formula (I), or a pharmaceutically acceptable salt thereof, is administered to the subject per day.

35. The compound for use of claim 31, wherein about 1200 mg of the compound of Formula (I), or a pharmaceutically acceptable salt thereof, is administered to the subject per day.

36. The compound for use of claim 31, wherein about 1600 mg of the compound of Formula (I), or a pharmaceutically acceptable salt thereof, is administered to the subject per day.

37. The compound for use of claim 1, further comprising administering to the subject one or more additional agent.

38. The compound for use of claim 37, wherein the additional agent is an angiogenesis inhibitor, an anti-PD-1 antibody, or an anti-PD-L1 antibody.

39. The compound for use of claim 38, wherein the additional agent is an anti-PD-1 antibody, wherein the anti-PD-1 antibody is cemiplimab, nivolumab, pembrolizumab, pidilizumab, spartalizumab, camrelizumab, sintilimab, tislelizumab, toripalimab, dostarlimab, sasanlimab, retifanlimab, tebotelimab, ABBV-181, AK104, AK105, BCD-100, BI-754091, CBT-501, CC-90006, GLS-010, HLX10, IBI-308, JNJ-3283, JS001, LZM009, MEDI0680 (AMP-514), REGN-2810, SHR-1210, Sym021, TSR-042, or XmAb20717.

40. The compound for use of claim 39, wherein the anti-PD-1 antibody is pembrolizumab.

41. The compound for use of claim 38, wherein the additional agent is an anti-PD-L1 antibody, wherein the anti-PD-L1 antibody is atezolizumab, avelumab, durvalumab, envafolimab, FS118, BCD-135, BGB-A333, BGBA-317, CBT-502, CK-301, CS1001, FAZ053, MDX-1105, MSB2311, SHR-1316, M7824, LY3415244, CA-170, or CX-072.

42. The compound for use of claim 41, wherein the anti-PD-L1 antibody is atezolizumab.

43. The compound for use of claim 38, wherein the additional agent is an angiogenesis inhibitor, wherein the angiogenesis inhibitor is bevacizumab, sorafenib, sunitinib, nilotinib, pazopanib, dasatinib, regorafenib, cabozantinib, lenvatinib, ponatinib, ziv-aflibercept, axitinib, tivozanib, everolimus, lenalidomide, thalidomide, vandetanib, orvandetanib, or ramucirumab.

44. The compound for use of claim 43, wherein the angiogenesis inhibitor is bevacizumab.

45. The compound for use of claim 37, wherein the additional agents are angiogenesis inhibitor and anti-PD-L1 antibody.

46. The compound for use of claim 45, wherein the additional agents are a atezolizumab and bevacizumab.

47. A compound of Formula (II): 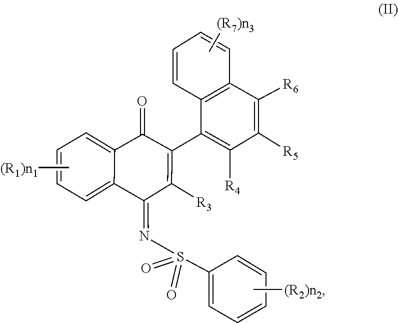
or a pharmaceutically acceptable salt thereof, for use in a method of treating a subject diagnosed with a non-viral liver cancer, the method comprising administering to the subject an effective amount of the compound of Formula (II), or a pharmaceutically acceptable salt thereof, wherein
each occurrence of R₁ is independently hydrogen, halogen, cyano, nitro, CF₃, OCF₃, ORa, SRa, C(═O)Ra, OC(═O)Ra, C(═O)ORa, NRbRc, NRbC(═O)Rc, C(═O)NRbRc, NRbC(═O)ORc, OC(═O)NRbRc, NRaC(═O)NRbRc, alkyl, alkenyl, cycloalkyl, optionally substituted aryl, or optionally substituted heterocycle;
n₁ is 0, 1, 2, 3, or 4;
each occurrence of R₂ is independently hydrogen, halogen, cyano, nitro, CF₃, OCF₃, ORa, SRa, C(═O)Ra, OC(═O)Ra, C(═O)ORa, NRbRc, NRbC(═O)Rc, C(═O)NRbRc, NRbC(═O)ORc, OC(═O)NRbRc, NRaC(═O)NRbRc, alkyl, alkenyl, cycloalkyl, cycloalkenyl, optionally substituted aryl, optionally substituted aryloxyl, or optionally substituted heterocycle;
n₂ is 0, 1, 2, 3, 4, or 5;
R₃ is hydrogen, halogen, cyano, nitro, CF₃, OCF₃, ORa, SRa, OC(═O)Ra, alkyl, alkenyl, cycloalkyl, or optionally substituted aryl or heteroaryl;
R₄ is hydrogen, halogen, cyano, nitro, CF₃, OCF₃, ORa, SRa, NRbRc, OC(═O)Ra, alkyl, alkenyl, or cycloalkyl;
each occurrence of R₅, R₆, and R₇ is independently hydrogen, halogen, cyano, nitro, CF₃, OCF₃, ORa, SRa, C(═O)Ra, OC(═O)Ra, C(═O)ORa, NRbRc, NRbC(═O)Rc, C(═O)NRbRc, NRbC(═O)ORc, OC(═O)NRbRc, NRaC(═O)NRbRc, alkyl, alkenyl, cycloalkyl, optionally substituted aryl, or optionally substituted heterocycle;
n₃ is 0, 1, 2, 3, or 4; and
each occurrence of Ra, Rb, and Rc is independently hydrogen, alkyl, cycloalkyl, alkenyl, cycloalkenyl, alkynyl, heterocycle, or aryl; or said Rb and Rc together with the nitrogen atom to which they are bonded optionally form a heterocycle comprising 1-4 heteroatoms.

48. The compound for use of claim 47, wherein the compound of Formula (II) is a compound of Formula (III): 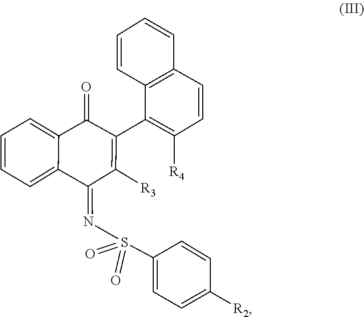
or a pharmaceutically acceptable salt thereof.

49. The compound for use of claim 48, wherein the compound of Formula (III) is a compound of Formula (IV): 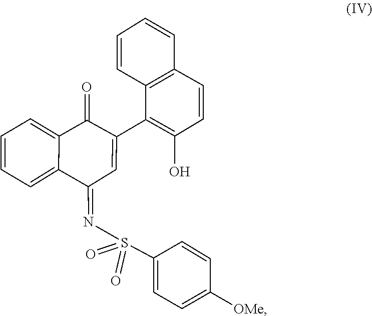 [SMILES: COc1ccc(S(=O)(=O)/N=C2\C=C(c3c(O)ccc4ccccc34)C(=O)c3ccccc32)cc1]
or a pharmaceutically acceptable salt thereof.

50. The compound for use of claim 47, wherein the non-viral liver cancer is hepatocellular carcinoma, fibrolamellar carcinoma, bile duct cancer, angiosarcoma, or hepatoblastoma.

51. The compound for use of claim 47, wherein the non-viral liver cancer is hepatocellular carcinoma.

52. The compound for use of claim 47, wherein the subject has alcoholic liver disease, alcoholic cirrhosis, autoimmune disease, metabolic disease, alcoholic steatohepatitis, non-alcoholic fatty liver disease, non-alcoholic steatohepatitis, diabetes, obesity, iron overload syndrome, autoimmune hepatitis, primary biliary cholangitis, cryptogenic liver disease, alcohol use, tobacco use, oral contraceptive use, pesticide exposure, aflatoxin exposure, or betel quid chewing.

53. The compound for use of claim 47, wherein the subject is negative for hepatitis B virus.

54. The compound for use of claim 47, wherein the subject is negative for hepatitis B surface antigen.

55. The compound for use of claim 47, wherein the subject is negative for hepatitis C virus.

56. The compound for use of claim 47, wherein the subject is negative for anti-HCV antibody.

57. The compound for use of claim 47, wherein the subject has been administered one or more prior cancer therapy.

58. The compound for use of claim 57, wherein the prior cancer therapy is sorafenib, pembrolizumab, nivolumab, lenvatinib, bevacizumab, atezolizumab, ramucirumab, cabozantinib, infigratinib, pemigatinib, regorafenib, tremelimumab, or durvalumab, or pharmaceutically acceptable salts thereof, or any combination thereof.

59. The compound for use of claim 57, wherein the prior cancer therapy is sorafenib, pembrolizumab, nivolumab, lenvatinib, or bevacizumab.

60. The compound for use of claim 47, wherein the non-viral liver cancer has progressed from, or the subject was intolerant to, a prior first line cancer therapy.

61. The compound for use of claim 47, wherein the non-viral liver cancer has progressed from, or the subject was intolerant to, a prior second line cancer therapy.

62. The compound for use of claim 47, wherein the non-viral liver cancer has progressed from, or the subject was intolerant to, a prior third line cancer therapy.

63. The compound for use of claim 47, wherein about 1 mg/kg/day to about 50 mg/kg/day of the compound of Formula (II), or a pharmaceutically acceptable salt thereof, is administered to the subject.

64. The compound for use of claim 63, wherein about 5 mg/kg/day to about 30 mg/kg/day of the compound of Formula (II), or a pharmaceutically acceptable salt thereof, is administered to the subject.

65. The compound for use of claim 47, wherein about 200 mg to about 2000 mg of the compound of Formula (II), or a pharmaceutically acceptable salt thereof, is administered to the subject per day.

66. The compound for use of claim 47, further comprising administering to the subject one or more additional agent.

67. The compound for use of claim 47, wherein the additional agent is an angiogenesis inhibitor, an anti-PD-1 antibody, or an anti-PD-L1 antibody.

68. The compound for use of claim 67, wherein the additional agent is an anti-PD-1 antibody, wherein the anti-PD-1 antibody is cemiplimab, nivolumab, pembrolizumab, pidilizumab, spartalizumab, camrelizumab, sintilimab, tislelizumab, toripalimab, dostarlimab, sasanlimab, retifanlimab, tebotelimab, ABBV-181, AK104, AK105, BCD-100, BI-754091, CBT-501, CC-90006, GLS-010, HLX10, IBI-308, JNJ-3283, JS001, LZM009, MEDI0680 (AMP-514), REGN-2810, SHR-1210, Sym021, TSR-042, or XmAb20717.

69. The compound for use of claim 68, wherein the anti-PD-1 antibody is pembrolizumab.

70. The compound for use of claim 67, wherein the additional agent is an anti-PD-L1 antibody, wherein the anti-PD-L1 antibody is atezolizumab, avelumab, durvalumab, envafolimab, FS118, BCD-135, BGB-A333, BGBA-317, CBT-502, CK-301, CS1001, FAZ053, MDX-1105, MSB2311, SHR-1316, M7824, LY3415244, CA-170, or CX-072.

71. The compound for use of claim 70, wherein the anti-PD-L1 antibody is atezolizumab.

72. The compound for use of claim 67, wherein the additional agent is an angiogenesis inhibitor, wherein the angiogenesis inhibitor is bevacizumab, sorafenib, sunitinib, nilotinib, pazopanib, dasatinib, regorafenib, cabozantinib, lenvatinib, ponatinib, ziv-aflibercept, axitinib, tivozanib, everolimus, lenalidomide, thalidomide, vandetanib, orvandetanib, or ramucirumab.

73. The compound for use of claim 72, wherein the angiogenesis inhibitor is bevacizumab.

74. The compound for use of claim 66, wherein the additional agents are angiogenesis inhibitor and anti-PD-L1 antibody.

75. The compound for use of claim 74, wherein the additional agents are a atezolizumab and bevacizumab.
